(19) United States
(12) Patent Application Publication
(10) Pub. No.: US 2023/0020401 A1
(43) Pub. Date: Jan. 19, 2023

(54) METABOLIC REPROGRAMMING OF IMMUNE CELLS TO ENHANCE THE EFFICACY OF PROPHYLACTIC AND THERAPEUTIC VACCINES

(71) Applicant: Sanford Burnham Prebys Medical Discovery Institute, La Jolla, CA (US)

(72) Inventors: Ashima SHUKLA, San Diego, CA (US); Anindya BAGCHI, San Diego, CA (US); Ashutosh TIWARI, San Diego, CA (US)

(21) Appl. No.: 17/773,775

(22) PCT Filed: Nov. 6, 2020
(86) PCT No.: PCT/US20/59519

§ 371 Date: May 2, 2022

Related U.S. Application Data
(60) Provisional application No. 62/933,225, filed on Nov. 8, 2019.

(51) Int. Cl. A61K 31/166; A61K 39/215; A61K 39/145

(52) U.S. Cl. CPC A61K 31/166; A61K 39/215; A61K 39/145; A61K 2039/55505

(57) ABSTRACT

Provided herein are compositions comprising a vaccine composition and an agent that triggers metabolic reprogramming of B cells and methods of using the agent that triggers metabolic reprogramming of B cells to increase effectiveness of the vaccine by increasing memory B cell population. One aspect of the disclosure includes a method of increasing the effectiveness of a vaccine in a subject, which comprises administering a B cell metabolic reprogramming agent to the subject in a dose and schedule configured to increase the effectiveness of the vaccine, wherein the subject is administered with the vaccine.

### CROSS-REFERENCE

The present application claims the benefit of U.S. Provisional Application 62/933,225, filed Nov. 8, 2019, which is incorporated herein by reference in its entirety.

### BACKGROUND

Germinal centers (GCs) are sites where mature B cells proliferate, differentiate, and mutate their antibody genes through somatic hypermutations during a normal immune response against any pathogen or antigen. Such matured B cells, upon receiving stimulus, migrate from a dark zone to a light zone to express antibodies on the cell surface and compete for survivals via interacting with follicular dendritic cells and/or follicular helper T cells. In addition, the mature B cells also receive differentiation signal as either to develop as memory B cells or antibody producing plasma cells. GC reaction develops high-affinity B cell receptor (BCRs) expressing memory B cells and antibody producing plasma cells.

BCR-induced signaling pathways govern the B cell activation and fate decisions, and such signaling pathways may be differentially regulated based on BCR Immunoglobulin (Ig) isotypes. However, the molecular mechanisms and modulating the differentially regulating BCR-induced signaling pathways have yet to be elucidated.

### SUMMARY OF THE DISCLOSURE

The present disclosure is related to compositions of and methods of using an agent that triggers metabolic reprogramming of B cells to increase immunity in a subject. One aspect of the disclosure includes a method of increasing the effectiveness of a vaccine in a subject, which comprises administering a B cell metabolic reprogramming agent to the subject in a dose and schedule configured to increase the effectiveness of the vaccine, wherein the subject is administered with the vaccine. In some embodiments, the B cell metabolic reprogramming agent is a mitochondria fission inhibitor. In some embodiments, the B cell metabolic reprogramming agent is an agent increasing mitochondrial mass and/or enhancing mitochondrial function. In such embodiments, the agent increasing mitochondrial mass and/or enhancing mitochondrial function is a Drp1 inhibitor. In such embodiments, the Drp1 inhibitor is Mdivi-1, dynasore, or dyngo 4a. Where the agent is Drp1 inhibitor or Mdivi-1, dynasore, or dyngo 4a, it is contemplated that the dose can be between 1.0-50.0 mg/kg. In some embodiments, the dose is about 2.5 mg/kg.

In some embodiments, the B cell metabolic reprogramming agent is administered concurrently with the vaccine. In such embodiments, it is contemplated that the agent can be an immune enhancer for the vaccine. Alternatively and/or additionally, the B cell metabolic reprogramming agent is administered at least a day after administering the vaccine. Alternatively and/or additionally, the B cell metabolic reprogramming agent is administered at least 2 days after administering the vaccine. Alternatively and/or additionally, the B cell metabolic reprogramming agent is administered a plurality of times in a regular interval after administering the vaccine.

In some embodiments, the effectiveness of the vaccine is increased by inhibiting mitochondrial mass decrease (or increasing mitochondrial mass), e.g., in IgG cells, IgG1 positive cells. Alternatively and/or additionally, the effectiveness of the vaccine is increased by increasing memory B cell population in the subject. Alternatively and/or additionally, the effectiveness of the vaccine is increased by increasing memory B cell precursor population in the subject. Alternatively and/or additionally, the effectiveness of the vaccine is increased by increasing replenishment of memory B cell population in the subject after rechallenge. In such embodiments, the memory B cell population can comprise IgG cells. Alternatively and/or additionally, the effectiveness of the vaccine is increased by increasing TFh cell population in the subject after rechallenge.

In some embodiments, the dose and schedule is sufficient to increase antigen-specific antibody titers at least 50% in the subject compared to a subject not receiving the agent after administering the vaccine. Alternatively and/or additionally, the dose and schedule is sufficient to increase antigen-specific antibody titers at least 50% in the subject compared to a subject not receiving the agent after rechallenge. Alternatively and/or additionally, the dose and schedule is sufficient to prevent decreased oxygen consumption of mitochondria in IgG cells in the subject. In some instances, the vaccine comprises a live-attenuated vaccine, an inactivated vaccine, a recombinant vaccine, a conjugate vaccine, a polysaccharide, a DNA-based vaccines, an RNA-based vaccines, or a toxoid vaccine. In some instances, the vaccine comprises an influenza vaccine or a SARS-CoV2 vaccine.

Another aspect of the disclosure includes a method of increasing immunity against an antigen in a subject having an immune response against the antigen, which comprises administering a B cell metabolic reprogramming agent to the subject in a dose and schedule effective to increase a secondary immune response upon re-exposure to the antigen compared to a subject not being administered with the B cell metabolic reprogramming agent. In some embodiments, the B cell metabolic reprogramming agent is a mitochondria fission inhibitor. In some embodiments, the B cell metabolic reprogramming agent is an agent increasing mitochondrial mass or enhancing mitochondrial function. In such embodiments, it is preferred that the agent increasing mitochondrial mass or enhancing mitochondrial function is a Drp1 inhibitor. In such embodiments, it is preferred that the Drp1 inhibitor is Mdivi-1, dynasore, or dyngo 4a. Where the agent is Drp1 inhibitor or Mdivi-1, dynasore, or dyngo 4a, it is contemplated that the dose is between 1.0-50.0 mg/kg. In some embodiments, the dose is about 2.5 mg/kg.

In some embodiments, the agent is administered during the immune response. Alternatively and/or additionally, the schedule comprises administration at least a day after the immune response. Alternatively and/or additionally, the schedule comprises administration at least 2 days after the immune response. Alternatively and/or additionally, the schedule comprises administration a plurality of times in a regular interval after the immune response.

In some embodiments, the dose and schedule is sufficient to inhibit mitochondrial mass decrease in immune cells or B cells (e.g., IgG cell, IgG1 positive cells, IgG2 positive cells, IgG3 positive cells, IgG4 positive cells, IgM cell, IgA, IgE, or IgD cells) of the subject. Alternatively and/or additionally, the dose and schedule is sufficient to increase memory B cell population in the subject. Alternatively and/or additionally, the dose and schedule is sufficient to increase memory B cell precursor population in the subject. Alternatively and/or additionally, the memory B cell population is increased by facilitating replenishment of memory B cells in the subject after the re-exposure to the antigen. In such embodiments, the memory B cell population may comprise IgG1 positive cells. In some embodiment, the memory B cell population can comprise any of IgG cells, IgG1 positive cells, IgG2 positive cells, IgG3 positive cells, IgG4 positive cells, IgM cell, IgA, IgE, or IgD cells. Alternatively and/or additionally, the memory B cell population is increased by increasing TFh cell population in the subject after re-exposure to the antigen. Alternatively and/or additionally, the dose and schedule is sufficient to increase antigen-specific antibody titers at least 50% in the subject compared to a subject not receiving the mitochondria fission inhibitor after re-exposure to the antigen. Alternatively and/or additionally, the dose and schedule is sufficient to prevent decreased oxygen consumption of mitochondria in immune cells or B cells (e.g., IgG cell, IgG1 positive cells, IgG2 positive cells, IgG3 positive cells, IgG4 positive cells, IgM cell, IgA, IgE, or IgD cells) in the subject.

Another aspect of the disclosure includes a method of increasing the memory B cell population in a subject having an immune response against the antigen, which comprises administering a B cell metabolic reprogramming agent to the subject in a dose and schedule effective to increase the memory B cell population after exposure to the antigen compared to a subject not being administered with the B cell metabolic reprogramming agent. In some embodiments, the B cell metabolic reprogramming agent is a mitochondria fission inhibitor. In some embodiment, the B cell metabolic reprogramming agent is an agent increasing mitochondrial mass or enhancing mitochondrial function In such embodiments, it is preferred that the B cell metabolic reprogramming agent is a is a Drp1 inhibitor. In such embodiments, it is preferred that the Drp1 inhibitor is Mdivi-1, dynasore, or dyngo 4a. Where the agent is Drp1 inhibitor or Mdivi-1, dynasore, or dyngo 4a, it is contemplated that the dose is between 1.0-50.0 mg/kg. In some embodiments, the dose is about 2.5 mg/kg.

In some embodiments, the agent is administered concurrently with the vaccine. In such embodiments, it is contemplated that the agent can be an immune enhancer for the vaccine. Alternatively and/or additionally, the agent is administered at least a day after administering the vaccine. Alternatively and/or additionally, the agent is administered at least 2 days after administering the vaccine. Alternatively and/or additionally, the agent is administered a plurality of times in a regular interval after administering the vaccine.

In some embodiments, the dose and schedule is sufficient to inhibit mitochondrial mass decrease in immune cells or B cells (e.g., IgG cell, IgG1 positive cells, IgG2 positive cells, IgG3 positive cells, IgG4 positive cells, IgM cell, IgA, IgE, or IgD cells) of the subject. Alternatively and/or additionally, the dose and schedule is sufficient to increase memory B cell precursor population in the subject. Alternatively and/or additionally, the memory B cell population is increased by facilitating replenishment of memory B cells in the subject after the re-exposure to the antigen. In such embodiments, the memory B cell population can comprises any of IgG cells, IgG1 positive cells, IgG2 positive cells, IgG3 positive cells, IgG4 positive cells, IgM cell, IgA, IgE, and/or IgD cells. In some embodiments, the memory B cell population comprises IgG cells. In some embodiments, the memory B cell population comprises IgG1 positive cells. Alternatively and/or additionally, the memory B cell population is increased by increasing TFh cell population in the subject after re-exposure to the antigen. Alternatively and/or additionally, the dose and schedule is sufficient to increase antigen-specific antibody titers at least 50% in the subject compared to a subject not receiving the mitochondria fission inhibitor after re-exposure to the antigen. Alternatively and/or additionally, the dose and schedule is sufficient to prevent decreased oxygen consumption of mitochondria in immune cells or B cells (e.g., IgG cell, IgG1 positive cells, IgG2 positive cells, IgG3 positive cells, IgG4 positive cells, IgM cell, IgA, IgE, and/or IgD cells) in the subject.

Another aspect of the disclosure includes a pharmaceutical composition, comprising a vaccine composition and a B cell metabolic reprogramming agent. The agent is present in the composition in a dose effective to increase effectiveness of the vaccine. In some embodiments, the agent that triggers metabolic reprogramming of B cells is a mitochondria fission inhibitor. In some embodiments, the agent that triggers metabolic reprogramming of B cells is an agent increasing mitochondrial mass or enhancing mitochondrial function. In such embodiments, it is preferred that the mitochondria fission inhibitor is a Drp1 inhibitor. In such embodiments, it is preferred that the Drp1 inhibitor is Mdivi-1, dynasore, or dyngo 4a. Where the agent is Drp1 inhibitor or Mdivi-1, dynasore, or dyngo 4a, it is contemplated that the dose is between 1.0-50.0 mg/kg. In some embodiments, the dose is about 2.5 mg/kg. In some embodiments, the agent is an immune enhancer for the vaccine.

In some embodiments, the dose is effective to inhibit mitochondrial mass decrease in IgG1 positive cells in a subject when administered. Alternatively and/or additionally, the dose is effective to increase memory B cell population in a subject when administered. Alternatively and/or additionally, the dose is effective to increase memory B cell precursor population in a subject in a subject when administered. Alternatively and/or additionally, the dose of the agent is effective to increase replenishment of memory B cell population in the subject after rechallenge. In such embodiments, the memory B cell population can comprise IgG cell, IgG1 positive cells, IgG2 positive cells, IgG3 positive cells, IgG4 positive cells, IgM cell, IgA, IgE, and IgD cells cells. Alternatively and/or additionally, the dose is effective to increase TFh cell population in the subject after rechallenge. Alternatively and/or additionally, the dose is effective to increase antigen-specific antibody titers at least 50% in a subject when administered, compared to a subject not receiving the composition. Alternatively and/or additionally, the dose is effective to prevent decreased oxygen consumption of mitochondria in IgG cell, IgG1 positive cells, IgG2 positive cells, IgG3 positive cells, IgG4 positive cells, IgM cell, IgA, IgE, or IgD cells in a subject when administered. In some instances, the vaccine composition comprises a live-attenuated vaccine, an inactivated vaccine, a recombinant vaccine, a conjugate vaccine, a polysaccharide, a DNA-based vaccines, an RNA-based vaccines, or a toxoid vaccine. In some instances, the vaccine composition comprises an influenza vaccine or a SARS-CoV2 vaccine.

Another aspect of the disclosure includes a pharmaceutical composition comprising i) a substance stimulating antibody production in a subject to provide immunity associated with a disease and ii) a mitochondria fission inhibitor.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the subject matter disclosed herein are set forth with particularity in the appended claims. A better understanding of the features and advantages of the subject matter disclosed herein will be obtained by reference to the following detailed description that sets forth illustrative embodiments, in which the principles of the subject matter disclosed herein are utilized, and the accompanying drawings of which:

FIG. 1 is a schematic of the germinal center reaction process.

FIG. 2 is a schematic showing distinct signaling potential by different B cell receptor (BCR) isotypes.

FIGS. 3A-B shows bar graphs indicating metabolic changes during an immune response and memory formation in B cells.

FIGS. 4A-D show data indicating that IgG1 expressing B cells have decreased mitochondrial mass.

FIGS. 5A-D show data indicating that IgM expressing B cells dominates the memory B cell pool.

FIGS. 6A-C show data indicating that IgG1 expressing B cell have more calcium flux and Drp-1 expression.

FIGS. 7A-B show data indicating that B cells expressing IgG1 undergoes metabolic reprogramming.

FIGS. 8A-B show small molecule screen to identify the molecules enhancing the oxphos of in vitro stimulated B cells.

FIGS. 9A-E show data indicating that incorporation of Mdivi-1 with immunization enhances the mitochondrial mass and survival of IgG1 GC B cells.

FIGS. 10A-B show data of mitochondrial mass of immune cells in vivo.

FIGS. 11A-C show data indicating the effect of Mdivi-1 on memory formation.

FIGS. 12A-D show data of Mdivi-1 effect on memory and antigen specific response.

FIGS. 13A-D show data indicating that Mdivi-1 inhibitor enhances antigen specific response.

FIG. 14 shows a graph of the NP specific antibody production measured by ELISA in control and Mdivi-1 treated group.

FIG. 15 shows a test immunization protocol.

FIG. 16 shows a graph of the NP specific antibody production measured by ELISA in control and Mdivi-1 treated group upon rechallenge with NP-CGG in PBS on Day 61.

FIGS. 17A-B show data indicating that Mdivi-1 treated mice replenished their memory B cell pool.

FIGS. 18A-B show effect of Mdivi-1 to memory B cells and Tfh cells.

FIG. 19 show effect of Mdivi-1 to memory precursor cells.

FIG. 20 shows another test immunization protocol.

FIGS. 21A-B show data indicating that IgG1 memory B cells are increased Mdivi-1 treated immunized mice.

FIGS. 22A-B show data indicating that IgG1 memory precursor cells are increased Mdivi-1 treated immunized mice.

FIGS. 23A-D show data indicating high expression of PD-1 molecule on T cells in Mdivi-1 treated group.

FIGS. 24A-B show metabolic alteration by Mdivi-1 increases the efficacy of immunization.

FIG. 25 shows confocal images of mitochondria organization in IgM and IgG1 cells with different treatments.

FIG. 26 shows flowmetry data of plasma cells (PC) differentiation in vitro.

FIG. 27 shows an experimental design to test immune enhancer effect on flu vaccine.

FIGS. 28A-B shows effect of Mdivi-1 to efficacy of flu vaccine.

FIG. 29 shows lung histology photographs representing disease progress post H1N1 infection.

FIG. 30A shows another experimental design to test immune enhancer effect on sheep red blood cell immunization.

FIG. 30B and FIG. 30C show scattered plots of antigen specific IgM (FIG. 30B) and antigen specific IgG1 (FIG. 30B) after immunization.

FIG. 31 illustrates an experiment schematic of SARS-CoV2 experiment to determine SBP-AS08 efficacy on SARS-CoV2 vaccine efficacy.

FIGS. 32A-D are graphs showing that SBP-AS08 enhances the vaccine specific cells, memory cells and antibody producing cells in mice.

FIGS. 33A-C show a schematic and data of development of a surrogate SARS-CoV2 Si Subunit Vaccine with SBP-AS08.

FIGS. 34A-C show data of antibody titers against S1 unit with SBP-AS08.

FIGS. 35A-B show an illustration and data of surrogate COVID-19 vaccine with SBP-AS08 generating more neutralizing antibodies.

FIGS. 36A-C show an illustration and data of developing novel neo-antigen vaccine based therapy for pancreatic cancer using SBP-AS08.

FIGS. 37A-B show survival rate graphs of tumor bearing mice.

FIG. 38A illustrates an experiment schematic of immunogen (SRBC) injection and SBP-AS08.

FIG. 38B shows a scattered plots of cells separated from harvested spleen of the animal and mitochondrial mass of isolated B cell and non-B cells.

### DETAILED DESCRIPTION OF THE INVENTION

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

“Treating” or “treatment” of a state, disorder or condition (e.g., cancer) includes: (1) preventing or delaying the appearance of clinical or sub-clinical symptoms of the disorder developing in a human that is afflicted with or pre-disposed to the disorder but does not yet experience or display clinical or subclinical symptoms of the disorder; and/or (2) inhibiting the disorder, including arresting, reducing or delaying the clinical manifestation of the disorder or at least one clinical or sub-clinical symptom thereof; and/or (3) relieving the disorder, e.g., causing regression of the disorder or at least one of its clinical or sub-clinical symptoms; and/or (4) causing a decrease in the severity of one or more symptoms of the disorder. The benefit to a subject to be treated is either statistically significant or at least perceptible to the patient or to the physician.

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some embodiments of any of the compositions and methods provided herein, “comprising” may be replaced with “consisting essentially of” or “consisting of.” The phrase “consisting essentially of” is used herein to require the specified feature(s) as well as those which do not materially affect the character or function of the claimed disclosure. As used herein, the term “consisting” is used to indicate the presence of the recited feature alone.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well of any dividual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well of any dividual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, “treatment of” or “treating,” “applying”, or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease or condition, or to a patient reporting one or more of the physiological symptoms of a disease, even though a diagnosis of this disease has not been made.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The section headings used herein are for organizational purposes only and are not to be construed as limiting the subject matter described.

Vaccination strategies, if purposed effectively, have the unprecedented ability to wipe-out diseases from the face of our planet. The success of any vaccine depends on its ability to generate robust immune response (generation of effector immune cells) and immunological memory, a phenomenon by which immune cells can vividly recall their previous encounters with a disease-causing agent to promptly attack it again. This is how a child receiving a hepatitis B vaccine, for example, remains immune to that disease throughout his/her life. While more and more vaccines against various infectious disease have been developed, the efficacy of such vaccines are not often satisfactory. For example, as shown in Table 1 (CDC expected estimates for 2019 influenza vaccine), flu vaccine effectiveness for the 2018-2019 flu season was estimated to be 61% against all flu types in children aged between 6 months and 17 years, and 24% for all flu types in adult patients 50 years and older. Yet, there are no agents or strategies marketed currently that are specifically designed to augment immunological memory in vaccine formulations, and consequentially, vaccine discovery continues to be an empirical and somewhat of a “hit and trial” process.

TABLE 1
Against influenza A or B viruses

| | Influenza | Influenza | Influenza | Influenza | | |
|---|---|---|---|---|---|---|
| Age group | positive | positive | negative | negative | Adjusted | Adjusted |
| (years) | Total | (% Vaccinated) | Total | (% Vaccinated) | VE % | 95% CI |
| All ages | 2795 | 48 | 7246 | 56 | 29% | (21 to 35) |
| 6 mos-8 | 759 | 40 | 1675 | 58 | 49% | (38 to 58) |
| 9-17 | 493 | 45 | 772 | 41 | 6% | (−22 to 27) |
| 18-49 | 831 | 39 | 2435 | 44 | 25% | (10 to 37) |
| 50-64 | 448 | 60 | 1324 | 62 | 12% | (−12 to 31) |
| ≥65 | 264 | 81 | 1040 | 83 | 12% | (−29 to 41) |

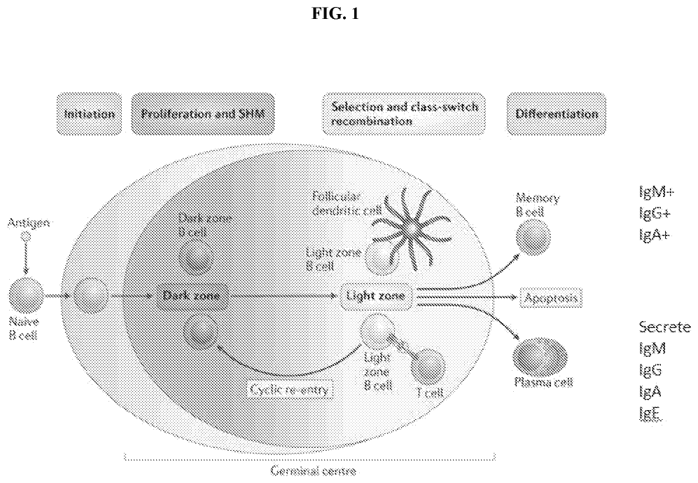

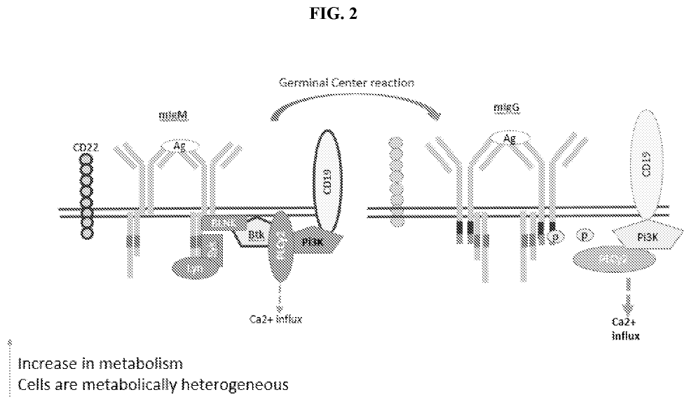

As illustrated in FIG. 1 (modified from: Bass & Dalla-Favera. Nature Reviews Immunology, 2015), Germinal center (GC) reaction develops high-affinity B cell receptor (BCRs) expressing memory B cells (IgG1 positive) and antibody producing plasma cells, which affinities increase over time to so induce affinity maturation of the antibodies. Simultaneously, antibody class switch may occur (e.g., from IgM to IgG, IgA, or IgE), as shown in FIG. 2, via class-switch recombination in the heavy chain of the antibodies such that antibodies can interact with different effectors for different functions (e.g., different signaling potential) without changing antigen specificity of the antibodies. Class switching to different antibody isotypes is regulated by B cell activation and cytokines via the BCRs. Such B cell differentiation is accompanied with cellular changes including increase of cell size, cellular organelle size (e.g., ER, secretory organelle), which is associated with increase of metabolism and generation of ATP.

The present disclosure relates to an agent that triggers metabolic reprogramming of B cells, and uses thereof to boost the immunity against an antigen or to boost the effect of a vaccine against the antigen by increasing the survival or population of antigen-specific memory B cells. Disclosed herein is that memory B cells have distinct mitochondrial mass and/or mitochondrial function when compared to germinal center cells and naïve B cells. Thus, a strategy can be developed to boost immunological memory and immune response by specific metabolic reprogramming of immune cells. In the present disclosure, a small molecule agents that acts as an immune enhancer to improve the efficacy of currently marketed or new feeble ineffective vaccines is used to enhance the efficacy and effectiveness of immune responses by increasing mitochondrial mass and mitochondrial function to so be used as a platform to improve the efficacy of several vaccines including prophylactic or therapeutic vaccines. Further disclosed herein is that inhibition of fatty acid oxidation in vitro augments the plasma cell differentiation, which are the primary antibody producing cells during immune responses. In one aspect of the disclosure, a method of increasing effectiveness of a vaccine in a subject by administering an agent that triggers metabolic reprogramming of B cells to the subject is disclosed. In this method, the agent that triggers metabolic reprogramming of B cells is administered to the subject, which previously had been administered with the vaccine.

In another aspect of the disclosure, a method of increasing immunity against an antigen in a subject having an immune response against the antigen is disclosed. In this method, an agent that triggers metabolic reprogramming of B cells is administered to the subject in a dose and schedule effective to increase a secondary immune response upon re-exposure to the antigen compared to a subject without administration of the agent that triggers metabolic reprogramming of B cells. As used herein the “secondary immune response” refers an immune response against an antigen that the subject had been previously exposed to. Thus, for example, where the first vaccination is administered to the subject and the second boost immunization (targeting the same antigen with the first vaccination) is administered to the subject 5 months after the first vaccination, any immune response against the antigen (e.g., generation of antigen-specific antibodies, increase the population of immune cells, etc.) after the second boost immunization would be deemed to be secondary immune response. Alternatively, in another example, where the first vaccination is administered to the subject and the subject is later exposed to the antigen (the same antigen targeted by the first vaccination), any immune response against the antigen (e.g., generation of antigen-specific antibodies, increase the population of immune cells, etc.) after the later antigen exposure would be deemed to be secondary immune response.

In another aspect of the disclosure, a method of increasing the memory B cell population in a subject having an immune response against the antigen is disclosed. In this method, an agent that triggers metabolic reprogramming of B cells is administered to a subject in a dose and schedule effective to increase the memory B cell population after exposure to the antigen compared to a subject without administration of the agent.

### Agent and Pharmaceutical Formulation

Any suitable agent that can trigger metabolic reprogramming of B cells are contemplated. In certain instances, the agent is an agent increasing mitochondrial mass or enhancing mitochondrial function. In some embodiments, such agent includes, but not limited to, a mitochondrial fission inhibitor or a mitochondrial complex-1 inhibitor (e.g., 3-(2,4-dichloro-5-methoxyphenyl)-2,3-dihydro-2-thioxo-4(1H)-quinazolinone, Mdivi-1), dynasore and dyngo 4a, a PKC inhibitor, Cal-101 (PI3K inhibitor), Etomoxir (fatty acid oxidation inhibitor), a mitochondrial fusion promoter, Rapamycin (mTOR inhibitor), a Gsk3 inhibitor, Fenofibrate (PPAR-alpha agonist), and BPTES (glutaminase inhibitor). In some embodiments, the agent that triggers metabolic reprogramming of B cells is a mitochondrial fission inhibitor that specifically inhibit the mitochondria fission. Mitochondrial fission is mediated by multiple pathways including RAS/RAF mediated ERK pathway, calcium-mediated pathway, glucose-mediated pathway, calmodulin-mediated pathway, hypoxia mediated pathway, starvation/energy-stress mediated pathway, SUMO-mediated pathway, which regulate the activity of DRP1 via phosphorylation on two serine residues. Activated DRP1 proteins are recruited by DRP1 receptors on the mitochondrial membrane, then assembled around the mitochondrial membrane to constrict the mitochondria. Thus, the suitable mitochondria fission inhibitors can include any Drp1 inhibitors or dynamin inhibitors. In some instances, the Drp1/dynamin inhibitor includes a chemical inhibitor, for example, Mdivi-1, dynasore and dyngo 4a. Alternatively and/or additionally, the Drp1/dynamin inhibitors include a nucleic acid such as inhibitory RNAs, for example, siRNA, RNAi, shRNA, etc. In some instances, the Drp1/dynamin inhibitor includes a peptide, such as a dominant negative forms of DRP1.

Thus, in some embodiments, the agents can be formulated as a pharmaceutical composition with a vaccine composition as an immune enhancer to boost the effectiveness of the vaccine composition. In some embodiments, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular, intra-arterial, intraperitoneal, intrathecal, intracerebral, intracerebroventricular, or intracranial) administration. In other instances, the pharmaceutical composition describe herein is formulated for oral administration. In still other instances, the pharmaceutical composition describe herein is formulated for intranasal administration. In some instances, the vaccine composition comprises live-attenuated vaccines (e.g., measles vaccine, rotavirus vaccine, smallpox vaccine, chickenpox vaccine, yellow fever vaccine, etc.), inactivated vaccines (e.g., flu vaccine, polio vaccine, Hepatitis A vaccine, rabies vaccine, etc.), subunit, recombinant, polysaccharide, and conjugate vaccines (e.g., Hib disease vaccine, Hepatitis B vaccine, whooping cough vaccine, pneumococcal disease vaccine, meningococcal disease vaccine, shingles vaccine, etc.), DNA-based vaccines, RNA-based vaccines, an influenza vaccine, a SARS-CoV2 vaccine, or toxoid vaccine (e.g., diphtheria vaccine, tetanus vaccine, etc.).

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some instances, the pharmaceutical formulation further includes pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulation further includes diluent which are used to solubilize and/or stabilize compounds because they provide a more stable environment. Salts dissolved in buffered solutions (which also provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as DiPac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some instances, the pharmaceutical formulation includes filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like. Stabilizers include compounds such as any antioxidation agents, buffers, acids, preservatives and the like.

### Therapeutic Regimens

In some embodiments, the pharmaceutical compositions described herein are administered for therapeutic applications. The agent that triggers metabolic reprogramming of B cells are administered to the subject in a dose and schedule effective to increase the effectiveness of the vaccine. In some embodiments, the vaccine comprises live-attenuated vaccines (e.g., measles vaccine, rotavirus vaccine, smallpox vaccine, chickenpox vaccine, yellow fever vaccine, etc.), inactivated vaccines (e.g., flu vaccine, polio vaccine, Hepatitis A vaccine, rabies vaccine, etc.), subunit, recombinant, polysaccharide, and conjugate vaccines (e.g., Hib disease vaccine, Hepatitis B vaccine, whooping cough vaccine, pneumococcal disease vaccine, meningococcal disease vaccine, shingles vaccine, etc.), DNA-based vaccines, RNA-based vaccines, an influenza vaccine, a SARS-CoV2 vaccine, or toxoid vaccine (e.g., diphtheria vaccine, tetanus vaccine, etc.). Thus, a dose and schedule of administering the agent may vary depending on the type of vaccines. In addition, a dose and schedule of administering the agent may vary depending on the type of the inhibitors (e.g., siRNA, RNAi, shRNA, miRNA, dominant negative forms of DRP1, Mdivi-1, dynasore, or dyngo 4a, etc.), or age, health condition, gender of the subject. Also, a dose and schedule of administering the agent may vary depending on any potential or expected known or unknown toxic effect to the subject.

For example, in embodiments that the agent is a mitochondrial fission inhibitor (e.g., Mdivi-1, dynasore, or dyngo 4a), the dose for administering to a subject (e.g., human) can be about 0.1-50 mg/kg, about 0.1-40 mg/kg, about 0.1-30 mg/kg, 0.1-20 mg/kg, about 0.2-15 mg/kg, about 0.5-15 mg/kg, about 1.0-50 mg/kg, about 1.0-40 mg/kg, about 1.0-30 mg/kg, about 0.5-10 mg/kg, about 0.8-10 mg/kg, about 1.0-10 mg/kg, about 1.0-9.0 mg/kg, about 1.0-8.0 mg/kg, about 1.0-7.0 mg/kg, about 1.0-6.0 mg/kg, about 1.0-5.0 mg/kg, about 0.5 mg/kg, about 1 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, or about 5.0 mg/kg, between 0.1-50 mg/kg, between 0.1-40 mg/kg, between 0.1-30 mg/kg, between 0.1-20 mg/kg, between 0.2-15 mg/kg, between 0.5-15 mg/kg, between 1.0-50 mg/kg, between 1.0-40 mg/kg, between 1.0-30 mg/kg, between 0.5-10 mg/kg, between 0.8-10 mg/kg, between 1.0-10 mg/kg, between 1.0-9.0 mg/kg, between 1.0-8.0 mg/kg, between 1.0-7.0 mg/kg, between 1.0-6.0 mg/kg, between 1.0-5.0 mg/kg, about 0.5 mg/kg, about 1 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 25.0 mg/kg, about 30.0 mg/kg, about 35.0 mg/kg, about 40.0 mg/kg, about 45.0 mg/kg, about 50.0 mg/kg. In some instances, the dose can be increased or decreased depending on the schedule of the administration. For example, where the mitochondrial fission inhibitor or the agent increasing or enhancing mitochondrial mass and mitochondrial function is Mdivi-1, dynasore, or dyngo 4a, the dose for administering to a subject (e.g., human) can be increased or decreased for about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, at least 0.01 mg/kg, at least 0.02 mg/kg, at least 0.03 mg/kg, at least 0.04 mg/kg, at least 0.05 mg/kg, at least 0.06 mg/kg, at least 0.07 mg/kg, at least 0.08 mg/kg, at least 0.09 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, or at least 1.0 mg/kg per each administration (e.g., for 3 consecutive administration, the dose can be increased from 2.0 mg/kg, 2.2 mg/kg, 2.4 mg/kg, respectively, or decreased from 2.4 mg/kg, 2.2 mg/kg, 2.0 mg/kg, respectively, etc.). In another example, where the mitochondrial fission inhibitor or the agent increasing or enhancing mitochondrial mass and mitochondrial function is Mdivi-1, dynasore, or dyngo 4a, the dose for administering to a subject (e.g., human) can be increased and then decreased, or decreased and then increased for about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, or about 1.0 mg/kg per each administration (e.g., for 5 consecutive administration, the dose can be increased from 2.0 mg/kg, 2.2 mg/kg, 2.4 mg/kg, then 2.2 mg/kg, and 2.0 mg/kg, respectively, etc.).

In some instances, the agent can be administered concurrently with the vaccine. In some instances, the agent can be administered at least within 10 min, within 30 min, within 1 hour, within 2 hours, within 3 hours, within 6 hours, within 12 hours after the vaccine administration. In some embodiments, the agent can be administered at least a day, at least 2 days, at least 3 days, at least 4 days, at least 5 days, at least 6 days, at least 7 days, at least 10 days, at least 12 days, at least 14 days, or at least 30 days after administering the vaccine. In some instances, where the vaccine administration schedule comprises a prime administration and a booster administration, the agent can be administered between the prime administration and the booster administration. Alternatively and/or additionally, in some embodiments, the agent can be administered a plurality of times in a regular interval after administering the vaccine. For example, the mitochondrial fission inhibitor can be administered once a day, once every two days, once every 3 days, once every 4 days, once every 5 days, once every 6 days, once every 7 days, once every 10 days, once every 14 days from day 0, day 2, day 3, day 4, day 5, day 6, day 7, day 10, day 14, day 28 after the administration of the vaccine. In some embodiments, the mitochondrial fission inhibitor or the agent increasing or enhancing mitochondrial mass and mitochondrial function can be administered at least once, at least twice, at least three times, at least four times, at least 5 times, at least 6 times, at least 7 times, at least 8 times, at least 9 times, or at least 10 times, during 0-60 days, 0-50 days, 0-45 days, 0-40 days, 0-30 days, 0-25 days, 0-20 days, or 0-15 days after the administration of the vaccine. Alternatively and/or additionally, in some embodiments, the mitochondrial fission inhibitor can be administered a plurality of times in an irregular interval, or increased interval, or decreased interval after administering the vaccine. For example, the mitochondrial fission inhibitor can be administered in two days increment (e.g., day 1, day 3, day 7, day 15, day 31 after the administration of the vaccine, etc.) or two days decrement (e.g., day 10, day 18, day 24, day 28, day 30 after the administration of the vaccine, etc.).

In certain instances, the mitochondria fission inhibitor or the agent increasing or enhancing mitochondrial mass and mitochondrial function is present in the pharmaceutical composition in a dose effective to increase effectiveness of the vaccine.

Thus, in certain instances, administration of the agent can be customized in a dose and/or a schedule to increase the effectiveness of the vaccine. The effectiveness of the vaccine can be determined, assessed, or predicted in various methods. For example, in some embodiments, the effectiveness of the vaccine can be determined by measuring the mitochondrial mass decrease in an immune cells, for example, an immune cell comprising an immunoglobulin isotype (e.g., IgG cell, IgG1 positive cells, IgG2 positive cells, IgG3 positive cells, IgG4 positive cells, IgM cell, IgA, IgE, IgD cells, or other immune cells). In some instances, the effectiveness of the vaccine can be determined by measuring the mitochondrial mass decrease in the immune cells. measuring the mitochondrial mass includes staining the mitochondria using the mitochondria-specific dye (e.g., MitoTracker Green, etc.) and measuring the fluorescent intensities of the mitochondria. In some instances, the effectiveness of the vaccine can be determined as being increased when the decrease of the mitochondrial mass in IgG cell, IgG1 positive cells, IgG2 positive cells, IgG3 positive cells, IgG4 positive cells, IgM cell, IgA, IgE, IgD cells, or other immune cells is inhibited significantly, e.g., at least 10%, at least 20%, at least 30%, at least 40%, at least 50%, at least 60%, at least 70%, at least 80%, at least 90%, etc, compared to the control (a patient or a condition that has not been treated with the mitochondrial fission inhibitor). Thus, for example, where the vaccine administration causes reduction of the mitochondrial mass in IgG cell, IgG1 positive cells, IgG2 positive cells, IgG3 positive cells, IgG4 positive cells, IgM cell, IgA, IgE, IgD cells, or other immune cells 50% higher than without vaccine administration, the dose and/or the schedule for administration of the mitochondrial fission inhibitor can be determined to achieve an effect of reduction of the mitochondrial mass in IgG cell, IgG1 positive cells, IgG2 positive cells, IgG3 positive cells, IgG4 positive cells, IgM cell, IgA, IgE, IgD cells, or other immune cells less than 40% higher, less than 30% higher, less than 20% higher, less than 10% higher, less than 5% higher than without vaccine administration.

Alternatively and/or additionally, the effectiveness of the vaccine can be determined by changes in memory B cell population in the subject. In some instances, measuring the mitochondrial mass can be performed using fluorescence-based or magnetic-based cell sorting methods (e.g., FACS, etc.). In certain instances, the effectiveness of the vaccine can be deemed increased when the immune cell population with any immunoglobulin isotypes (e.g., IgG1 positive memory B cell population (e.g., CD38+IgG1+ cells, antigen specific IgG1 cells, etc.)) is increased compared to the IgM positive memory B cell population (e.g., CD38+IgM cells, antigen specific IgM cells, etc.). Thus, for example, the effectiveness of the vaccine can be deemed increased when the immune cell population expressing IgG isotype (e.g., IgG1 positive memory B cell population (e.g., CD38+IgG1+ cells, antigen specific IgG1 cells, etc.)) is increased at least 10%, at least 20%, at least 30%, at least 40%, at least 50%, at least 60%, at least 70%, at least 80%, at least 90%, etc. Optionally, it is contemplated that the IgM positive memory B cell population (e.g., CD38+IgM+ cells, antigen specific IgM cells, etc.) is increased less than 50%, less than 40%, less than 30%, less than 20%, less than 10%. Alternatively and/or additionally, the effectiveness of the vaccine can be deemed increased when the ratio of increase in the IgG1 positive memory B cell population (e.g., CD38+IgG1+ cells, antigen specific IgG1 cells, etc.) and the increase in the IgM positive memory B cell population (e.g., CD38+IgM cells, antigen specific IgM cells, etc.) is more than 1:1, more than 3:2, more than 2:1, more than 3;1, more than 4:1, more than 5:1, etc. Thus, the dose and/or the schedule for administration of the mitochondrial fission inhibitor can be determined to achieve one or more of such effects that indicate the increase of the effectiveness of the vaccine.

Alternatively and/or additionally, the effectiveness of the vaccine can be determined by increased replenishment of memory B cell population in the subject after rechallenge. As used herein, the term “rechallenge” means exposure of the subject to an antigen that is targeted by the vaccine. Thus, the terms “rechallenge” and “re-exposure” can be interchangeably used. Also as used herein, the term “booster” means an additional vaccine administration after the primary administration (booster administration) of the vaccine after a certain interval. Thus, in some embodiments, the effectiveness of the vaccine can be deemed increased when the memory B cell population (e.g., IgG cell, IgG1 positive cells, IgG2 positive cells, IgG3 positive cells, IgG4 positive cells, IgM cell, IgA, IgE, IgD cells, IgG1 positive memory B cell (CD38+IgG1+) cells, antigen specific IgG1 cells, or other immune cells) is substantially or significantly increased. For example, the effectiveness of the vaccine can be deemed increased when the memory B cell population (e.g., preferably, IgG1 positive memory B cell (CD38+IgG1+) cells or antigen specific IgG1 cells) is increased significantly, in some embodiments, at least 10%, at least 20%, at least 30%, at least 40%, at least 50%, at least 60%, at least 70%, at least 80%, at least 90%, etc after 1 day, after 2 days, after 3 days, after 4 days, after 5 days, after 6 days, after 7 days, after 10 days of rechallenge, booster or re-exposure with the antigen or vaccine. Alternatively and/or additionally, in some embodiments, the effectiveness of the vaccine can be deemed increased when the memory B cell population (e.g., IgG1 positive memory B cell (CD38+IgG1+) cells or antigen specific IgG1 cells) is replenished at a level of at least 50%, at least 60%, at least 70%, at least 80%, at least 90%, at least 100% of the maximum or average number of memory B cell population (e.g., preferably, IgG1 positive memory B cell (CD38+IgG1+) cells or, antigen specific IgG1 cells) within 3 days, within 7 days, within 10 days after administrating the vaccine.

Alternatively and/or additionally, the effectiveness of the vaccine can be determined by increase of TFh cell population in the subject after rechallenge or booster. The effectiveness of the vaccine can be deemed increased when the TFh cell population is increased at least 10%, at least 20%, at least 30%, at least 40%, at least 50%, at least 60%, at least 70%, at least 80%, at least 90%, etc after 1 day, after 2 days, after 3 days, after 4 days, after 5 days, after 6 days, after 7 days, after 10 days of rechallenge compared to the subject's sample without mitochondrial fission inhibitor treatment.

Alternatively and/or additionally, the dose and/or schedule of the schedule for administration of the mitochondrial fission inhibitor can be determined to increase antigen-specific antibody titers after administering the vaccine. Thus, for example, the effectiveness of the vaccine can be deemed increased when the antigen-specific antibody titers in the subject's sample is increased at least 10%, at least 20%, at least 30%, at least 40%, at least 50%, at least 60%, at least 70%, at least 80%, at least 90%, etc after 1 day, after 2 days, after 3 days, after 4 days, after 5 days, after 6 days, after 7 days, after 10 days after administering the vaccine compared to a subject not receiving the agent after administering the vaccine.

Alternatively and/or additionally, the dose and/or schedule for administrating the mitochondrial fission inhibitor can be determined to increase antigen-specific antibody titers after rechallenge or booster. Thus, for example, the effectiveness of the vaccine can be deemed increased when the antigen-specific antibody titers in the subject's sample is increased at least 10%, at least 20%, at least 30%, at least 40%, at least 50%, at least 60%, at least 70%, at least 80%, at least 90%, etc., after 1 day, after 2 days, after 3 days, after 4 days, after 5 days, after 6 days, after 7 days, after 10 days after rechallenge or booster, compared to the antibody titer 1 hour, 6 hours, 12 hours, 24 hours, 2 days, etc., before the rechallenge or booster compared to a subject not receiving the agent after administering the vaccine.

In some embodiments, the dose and/or schedule for administering the mitochondrial fission inhibitor can be determined to those sufficient to prevent decreased oxygen consumption of mitochondria in IgG cell, IgG1 positive cells, IgG2 positive cells, IgG3 positive cells, IgG4 positive cells, IgM cell, IgA, IgE, IgD cells, IgG1 positive memory B cell (CD38+IgG1+) cells, antigen specific IgG1 cells, or other immune cells in the subject. In some instances, methods of measuring mitochondrial oxygen consumption include extracellular oxygen consumption assay (e.g., MitoXpressÂ® Xtra technology, mitochondrial oxygen tension (mitoPO2) and consumption (mitoVO2), etc.). Thus, in some embodiments, where the oxygen consumption of mitochondria in IgG cells or IgG1 positive cells is decreased at about 50%, the effectiveness of the vaccine can be deemed increased when the oxygen consumption rate is decreased less than 30%, less than 20%, less than 10%, less than 5% compared to the subject's cells without the agent administration. Alternatively and/or additionally, the effectiveness of the vaccine can be deemed increased when the oxygen consumption rate is decreased less than 90%, less than 80%, less than 70%, less than 60%, less than 50%, less than 40%, less than 30%, less than 20%, less than 10% of the highest decrease or average decrease of oxygen consumption rate after 1 day, after 2 days, after 3 days, after 4 days, after 5 days, after 6 days, after 7 days, after 10 days after administering the vaccine.

### Kits/Article of Manufacture

Disclosed herein, in certain embodiments, are kits and articles of manufacture for use with one or more of the compositions and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include target nucleic acid molecule described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

The inventors examined whether distinct Ig isotypes of BCR plays a role in regulating the metabolic rewiring of GC B cells. To explore this, the inventors examined the mitochondrial biogenesis of IgM and IgG1 expressing GC B cells in immunized wildtype B6 mice and observed a significant decrease in mitochondrial mass of IgG1 expressing GC B cells. To confirm if this was a BCR isotype driven phenotype, the inventors used primary B cells from IgG1(i) and IgM (i) transgenic mice, in which B cells developmentally express IgG1 or IgM isotypes, respectively. Similar to GC B cells, IgG1 expressing primary B cells showed significantly lower mitochondrial mass compared with IgM expressing cells. Moreover, metabolic analysis of IgG1 expressing B cells revealed decreased oxygen consumption rate. Mechanistically, IgG1 B cells showed increased levels of Drp1, low levels of Myc and mTOR signaling, molecules mediating mitochondrial biogenesis and fission. Upon treating mice with a Drp1 inhibitor, Mdivi-1(two days post immunization), the inventors observed a significant increase in mitochondrial mass and longevity of IgG1 memory B cells. Likewise, upon recall 100% of mice in Mdivi-1 treated group showed secondary response, whereas in the control group only 33% of mice responded. Additionally, the examples provided below suggests that inhibition of fatty acid oxidation in vitro augments the plasma cells differentiation. Overall, the inventors' finding suggests that distinct Ig isotype alters the metabolic regulation and mitochondrial biogenesis to regulate the fate of B cells during an immune response.

### Example 1

### B Cells Undergoes Metabolic Changes During an Immune Response and Memory Formation

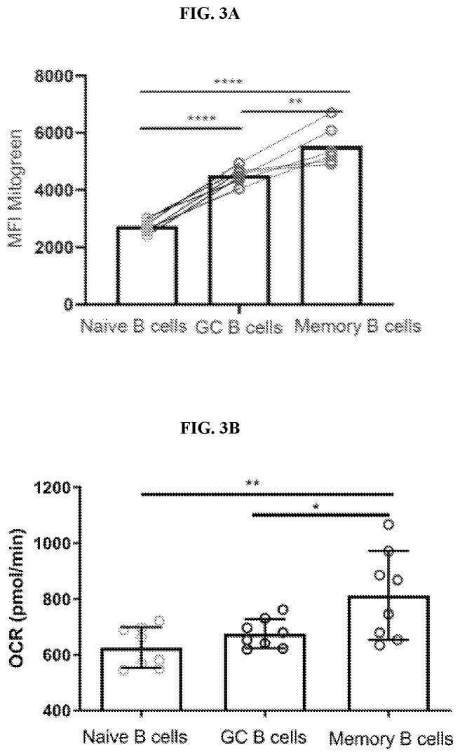

Six mice were immunized with 4-Hydroxy-3-nitrophenylacetyl-Chicken Gamma Globulin (NP-CGG) conjugated with aluminum adjuvant (Alum). Mice were sacrificed on Day 21 post immunization and spleen were harvested. Splenic cells were incubated Mitogreen dye to measure the mitochondrial mass at 37 C for 15 mins followed by surface staining of naïve B cells, germinal center (GC) B cells and Memory B cells using B220, NP-PE, CD38, Fas, IgD. FIG. 3A shows the bar graph of median fluorescence intensity (MFI) of Mitogreen in naïve, GC and memory B cells, each dot represents individual mice. Multiple t-tests were performed in prism grouped analysis p=0.01*, p=0.005**, p=0.0001***, p<0.0001****.

Nine mice were immunized with sheep red blood cells (SRBC) on Day 0 and Day 4. On Day 15 post immunization mice were injected with BSA conjugated with palmitate 40 mins before sacrificing mice. Mice were sacrifice and spleens were harvested and pooled in 3 mice per group. Naive, GC, Memory B cells were MACS using CD43, CD11 c; CD43, CD38, IgD, CD11c and CD43, GL7, CD11c, IgD. Cells were plated in 1.5 million per well in triplicates and seahorse assay was performed. FIG. B shows a bar graph of a basal oxygen consumption rate (OCR) of cells. Multiple t-tests were performed in prism grouped analysis p=0.01*, p=0.005**.

### Example 2

### IgG1 Expressing B Cells Have Decreased Mitochondrial Mass

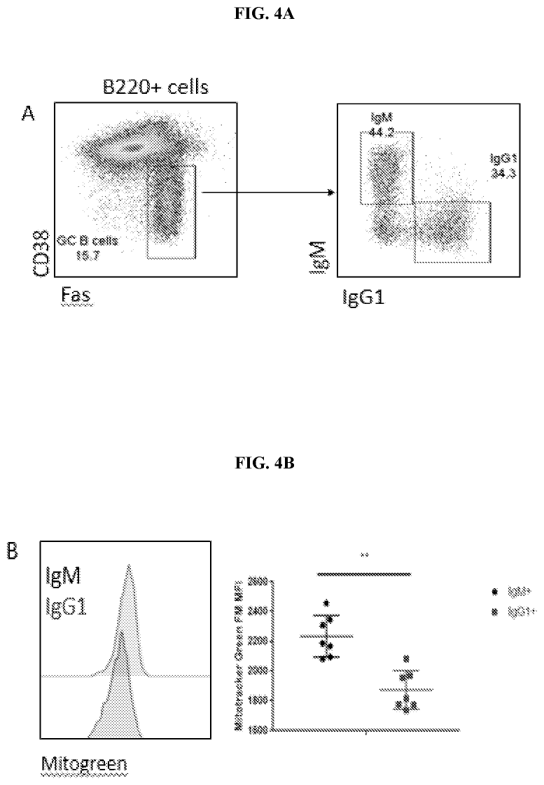

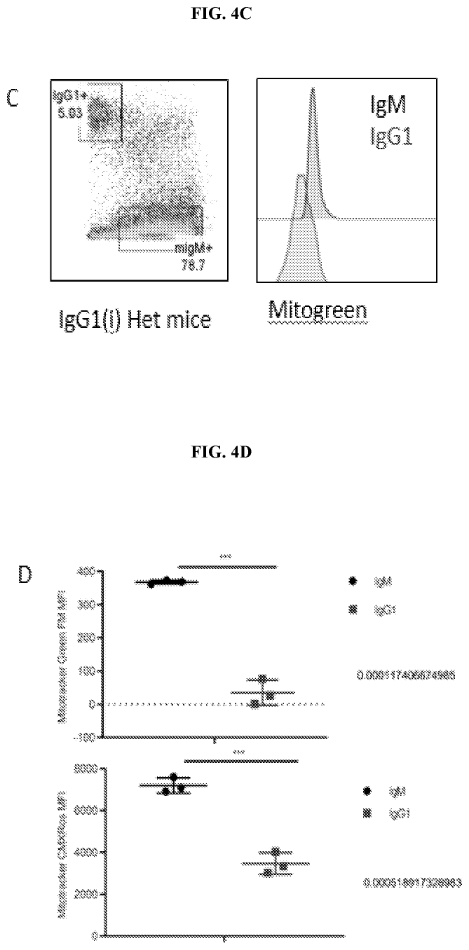

The inventors examined the mitochondrial biogenesis of IgM and IgG1 expressing GC B cells in immunized wildtype C57B6 mice to determine whether distinct Ig isotypes of B cell receptor (BCR) plays a role in regulating the metabolic rewiring of GC B cells. 8 weeks old wild type C57B6 mice were immunized with sheep red blood cells (SRBCs) via intraperitoneal injections two times on Day 0 and Day 5, respectively. Then the blood content of the mice was analyzed on Day 11. FIG. 4A is a flow plot that shows CD38lowFas+ germinal center (GC) B cells in spleen, further gated on IgM and IgG1 GC B cells. FIG. 3B shows a histogram plot determining the levels of mitochondria using Mitogreen tracker in GC IgM and IgG1 cells (p<0.001). Right side in FIG. 4B is a graph showing median fluorescent intensity (MFI) of Mitogreen tracker in GC IgM and IgG1 cells n=7 mice. To confirm if this was a BCR isotype driven phenotype, primary B cells from IgG1(i) and IgM (i) transgenic mice, in which B cells developmentally express IgG1 or IgM isotypes, respectively, were used. Similar to GC B cells, IgG1 expressing primary B cells showed significantly lower mitochondrial mass compared with IgM expressing cells. FIG. 4C shows a flow plot of IgM and IgG1 staining (left) and histogram of Mitogreen levels in IgM and IgG1 cells (right) from B cells isolated from IgG1(i) knock-in transgenic mice. FIG. 4D shows a graph showing MFI values of Mitogreen (top) and Mitotracker CMXRos (bottom) in n=3 mice. This example shows that decrease of mitochondrial mass is more prevalent in IgG1 B cells than IgM B cells, indicating that IgG1 B cells are more susceptible in selective cell death upon immunization.

### Example 3

### IgM Expressing B Cells Dominates the Memory B Cell Pool

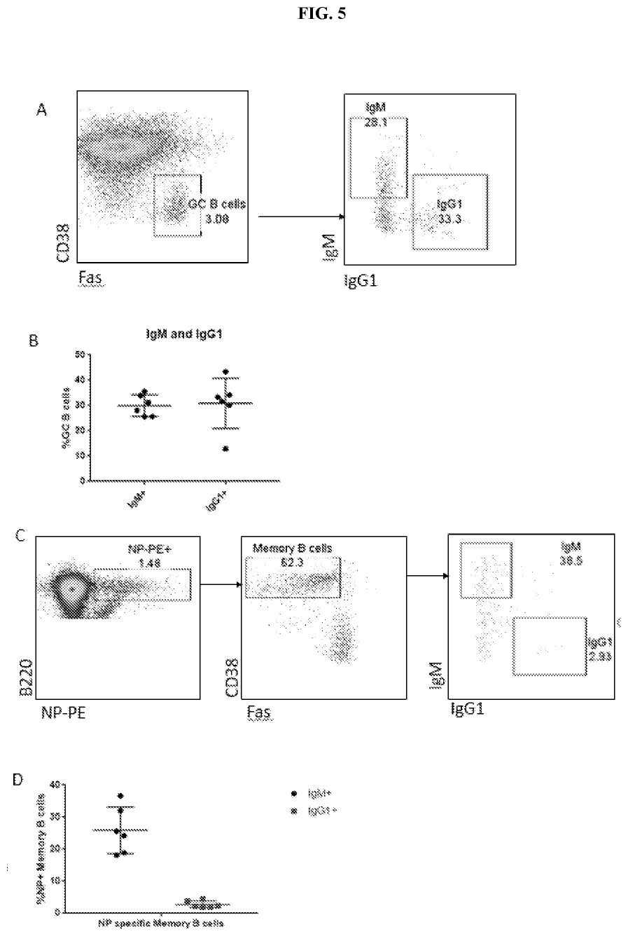

8 weeks old wild type C57B6 mice were immunized with NP-CGG conjugated with Alum, and then the blood samples from the immunized mice were analyzed on Day 14 post immunization. FIG. 5A shows a flow plot showing CD38lowFas+ germinal center (GC) B cells in spleen, further gated on IgM and IgG1 GC B cells. FIG. 5B shows frequencies of IgM and IgG1 cells in GCs. FIG. 5C shows a flow plot showing NP specific B cells, memory B cells, IgM and IgG1 memory cells. FIG. 5D shows a graph displaying the frequencies of IgM and IgG1 NP specific memory B cells. This example shows that upon immunization, IgM type memory B cells are selectively survived or proliferated such that IgM type memory B cells dominates the memory B cell pools over IgG1 type memory B cells.

### Example 4

### IgG1 Expressing B Cell Have More Calcium Flux and Drp-1 Expression

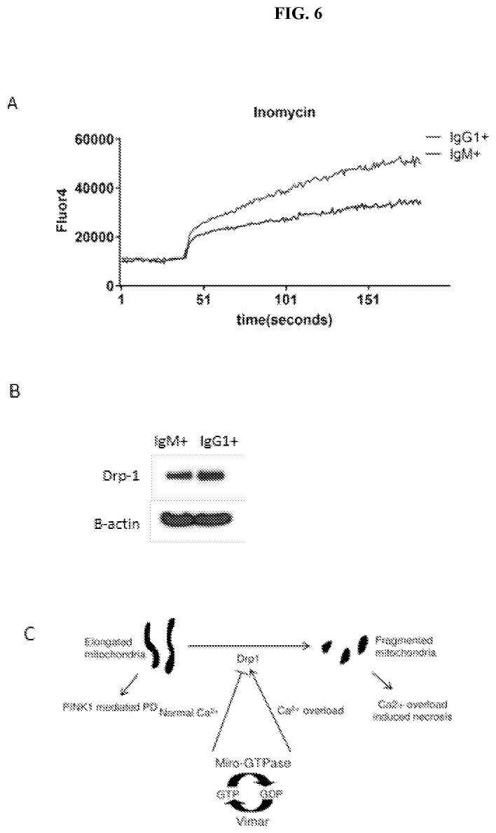

Metabolic analysis of IgG1 expressing B cells revealed decreased oxygen consumption rate. Mechanistically, IgG1 B cells showed increased levels of Drp1, a critical molecule mediating mitochondrial biogenesis and fission. IgM and IgG1 cells were isolated from IgG1(i) and IgM(i) transgenic mice. Cells were stained with Fluor4 AM at 37 degree water bath for one hour. The calcium flux upon inomycin treatment to measure the maximum calcium flux intensity. As shown in FIG. 6A, calcium flux was significantly higher in IgG1 B cells compared to IgM B cells. FIG. 6B is the immunoblot of anti-Drp1 and beta actin as loading control in IgM and IgG1 B cells at basal level, indicating that Drp-1 expression was also substantially higher in IgG1 B cells compared to IgM B cells. FIG. 6C shows schematic showing the regulation of Drp-1 by Calcium signaling (Ding et al., PLOS Genetics, 2016). This example indicates that loss of IgG1 (or cell death of IgG1, compared to IgM cells) is associated with higher expression of Drp1 in the IgG1 B cells, and such Drp-1 activity is further facilitated by higher calcium flux in the IgG1 B cells, which results in mitochondrial fragmentation in the IgG1 B cells.

### Example 5

### B Cells Expressing IgG1 Undergoes Metabolic Reprogramming

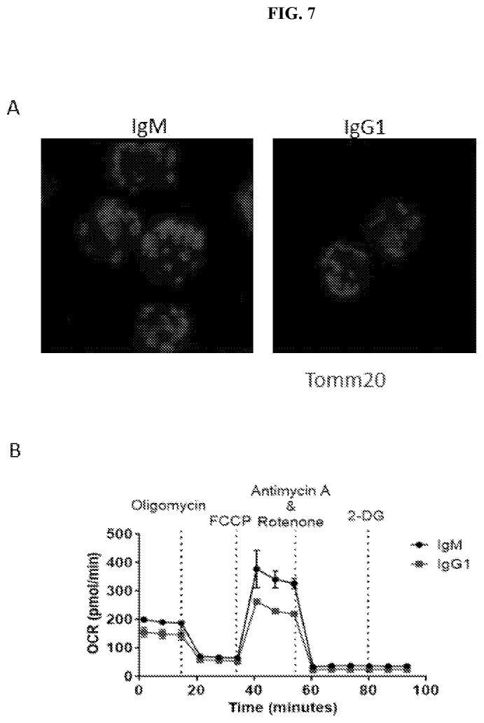

IgM and IgG1 cells were isolated with from IgM(i) and IgG1(i) transgenic mice, and the mitochondria in IgM and IgG1 cells were visualized by staining the cell with Tomm20. FIG. 7A shows confocal images of IgM and IgG1 expressing B cells stain with antibody against Tomm20 to label mitochondria. FIG. 7B shows a graph of Oxygen consumption rate measured during mitochondria stress test using seahorse bioanalyzer. This example indicates that IgG1 B cell receptor leads to metabolic reprogramming of B cells.

### Example 6

### A Small Molecule Screen to Identify the Molecules Enhancing the Oxphos of In Vitro Stimulated B Cells

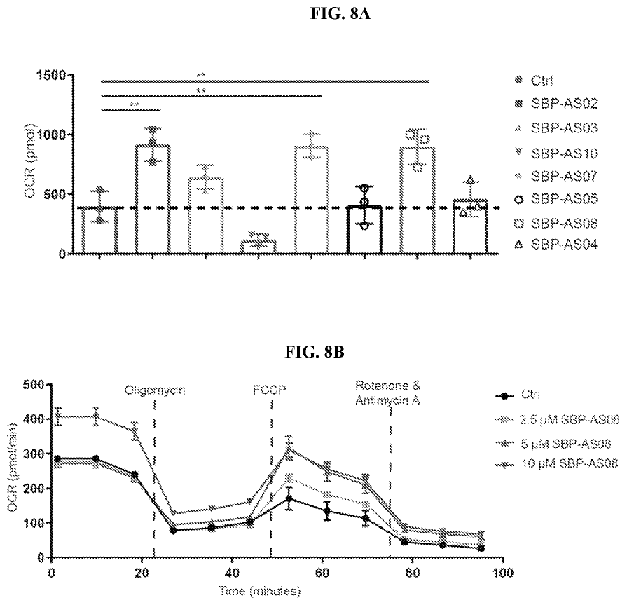

Naïve B cells were stimulated with CD40 and anti-IgM for 24 hours. After 24 hours molecules were added M1 fusion promoter as SBP-AS02, Dynasore as SBP-AS03, Dyngo-4a as SBP-AS10, Rapamycin as SBP-AS07, Hemin as SBP-AS05, Mdivi-1 as SBP-AS08 and Bafilomycin as SBP-AS04. At 48 hours, mitostress test using seahorse bioanalyzer was performed to determine the oxygen consumption rate (OCR), cells were plated in triplicates. FIG. 8A is a bar graph of basal OCR in each condition. FIG. 8B shows a line graph of the time lapse oxygen consumption rate in various concentrations of Mdivi-1 (SBP-A508). Mdivi-1 was added to cells at the concentration of 2.5 μM, 5 μM and 10 μM, and mitostress test using seahorse bioanalyzer was performed to determine the OCR.

### Example 7

### Incorporation of Mdivi-1 with Immunization Enhances the Mitochondrial Mass and Survival of IgG1 GC B Cells

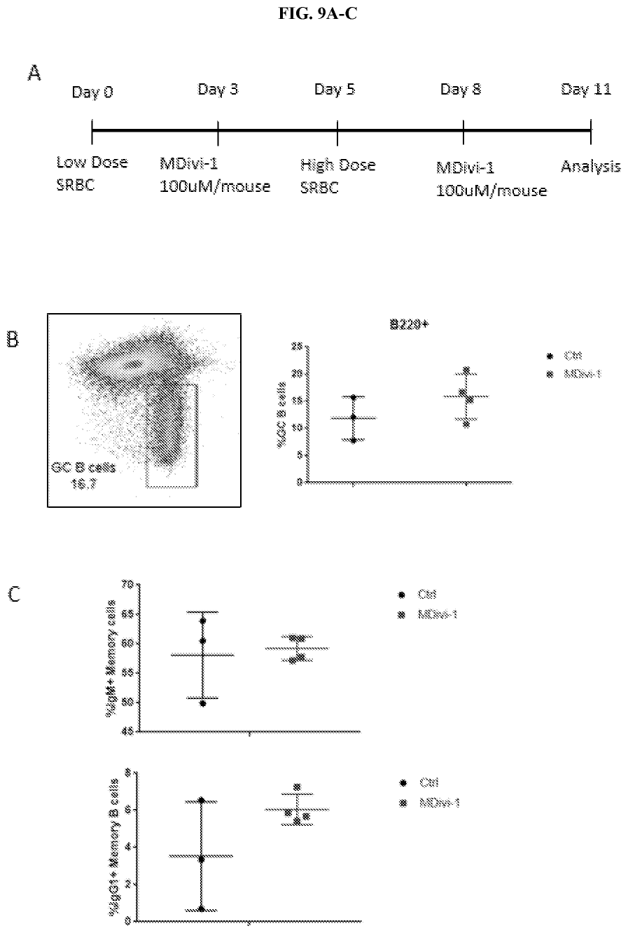

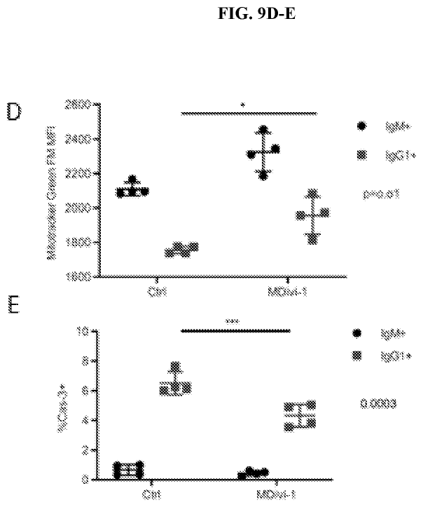

Upon treating of mice with a Drp1 inhibitor, Mdivi-1 (two days post immunization), a significant increase in IgG1 memory B cells was observed. FIG. 9A shows a schematic of experimental design of immunization and Mdivi-1 treatment to the 8 weeks old C57B6 mice. Briefly, mice were treated with Mdivi-1 twice, each 3 days after the low or high dose of Sheep Red Blood Cells (SRBC) administration (as an antigen). FIG. 9B shows a flow plot and a graph of frequencies of GC B cells in control and Mdivi-1 treated group, indicating GC B cells were increased with Mdivi-1 treatments. FIG. 9C shows a graph of frequencies of IgM (top) and IgG1 (bottom) expressing memory cells. FIG. 9D shows a graph of mitochondrial mass measured by Mitotracker green MFI in IgM and IgG1 GC B cells in control and Mdivi-1 treated mice group. FIG. 9E shows a graph of survival measured by active caspase-3 using flow cytometry in IgM and IgG1 GC B cells in control and Mdivi-1 treated mice group. This example indicates that Mdivi-1 treatment prevents IgG1 B cell loss through mitochondrial fragmentation and apoptosis, thereby increasing the IgG1+ memory B cell pools upon immunization.

### Example 8

### SBP-AS08 Enhances the Mitochondrial Mass of Immune Cells In Vivo

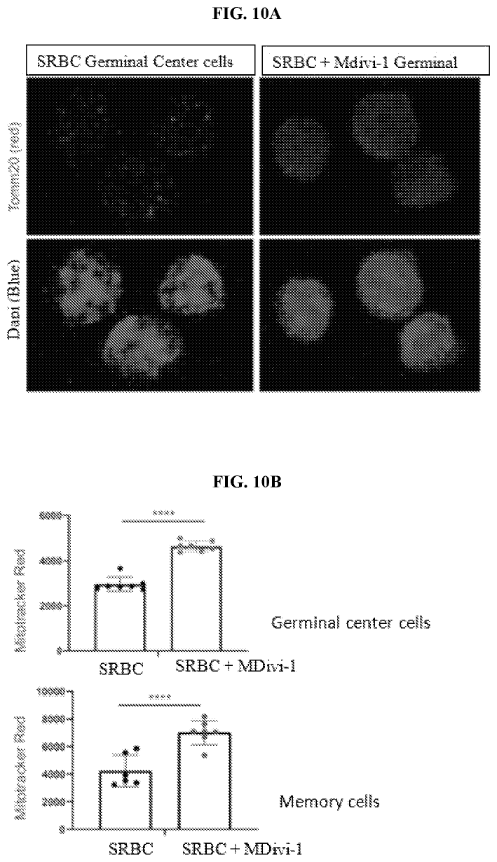

Seven mice each in two group were immunized with SRBC on Day 0 and Day 4. One group on Day 3 and Day 7 were given SBP-A508 at 2.5 mg/kg. GC B cells were MACS and stained with Tomm20 (red) to stain mitochondria and DAPI to stain nucleus on Day 15. Cells were imaged using confocal microscope. FIG. 10A shows the images of GC B cells from mice immunized with SRBC only and SRBC with SBP-AS08. FIG. 10B shows bar graphs of mitochondrial mass measured by mitotracker red MFI in GC B cells and Memory B cells in control and SBP-A508 treated mice group. This data indicates incorporation of SBP-A508 with immunization enhances the mitochondrial mass of germinal center B cells and memory B cells.

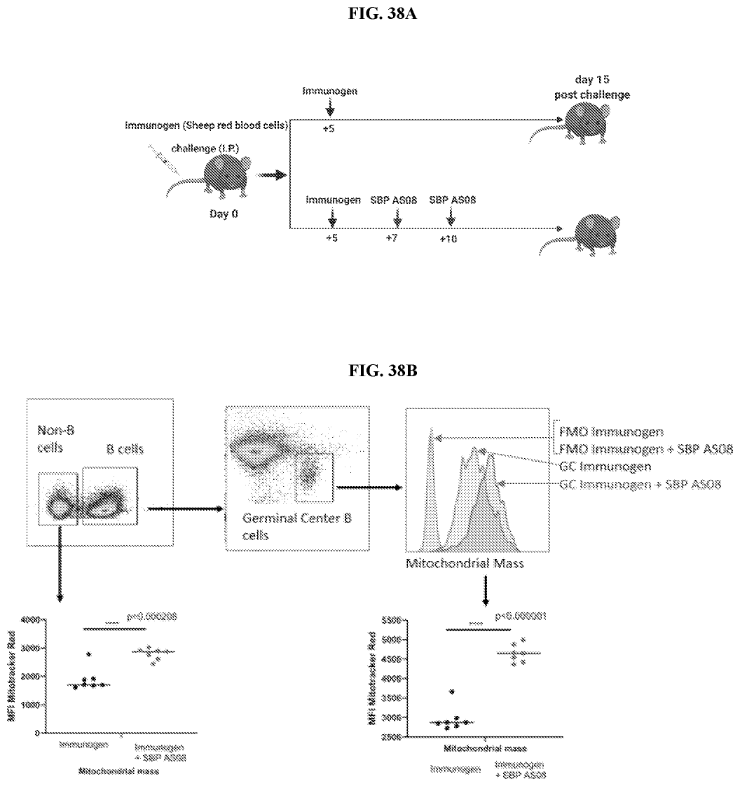

In another experiment, mice each in two group were immunized with SRBC on Day 0 and Day 5. One group of mice was also injected with SBP-A508 on Day 7 and Day 10. One a to induce humoral immune responses. As shown in the experimental scheme in FIG. 38A, one group of mice were inje Day 15 post immunization, mice were sacrificed and spleens were harvested. Cells were incubated in Mitotracker red dye 200 nM per ml of RPMI for 15 mins at culture conditions. Cells were further stained with surface antibodies B220, CD38, Fas to determine B cells, Germinal Center and non-B cells populations. As shown in FIG. 38B, mitochondrial mass of both B cells and non-B cells were significantly increased with SBP-AS08 administration.

### Example 9

### Effect of Mdivi-1 on Memory Formation

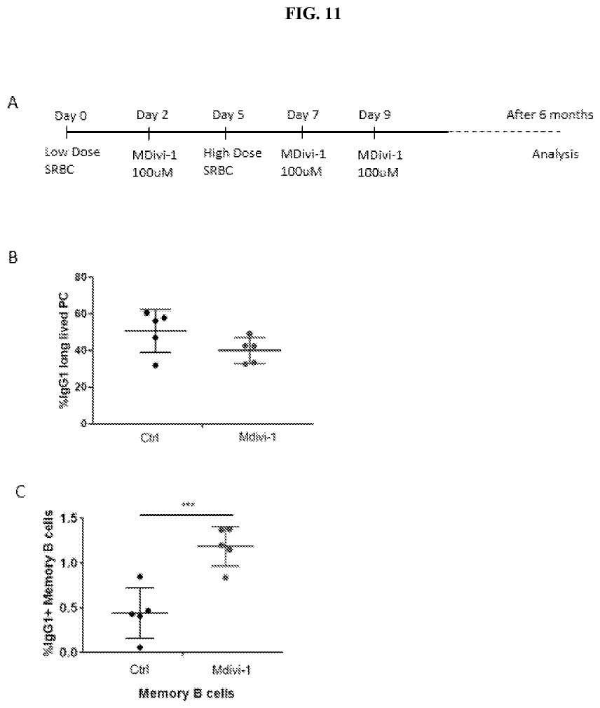

FIG. 11A shows a schematic of an experimental design to study the effect of Mdivi-1 on IgG1 memory cells formation after 6 months post immunization. FIG. 11B shows a graph displaying the frequencies of long lived IgG1 producing plasma cells in bone marrow. FIG. 11C shows a graph displaying the frequencies of IgG1 expressing memory B cells in spleen. This example indicates that the increase of IgG1 positive B cells upon Mdivi-1 treatment is specific to the memory B cells, confirming that the Mdivi-1 treatment facilitates immune memory formation.

### Example 10

### SBP-AS08 Promotes More Memory and Effector Immune Responses

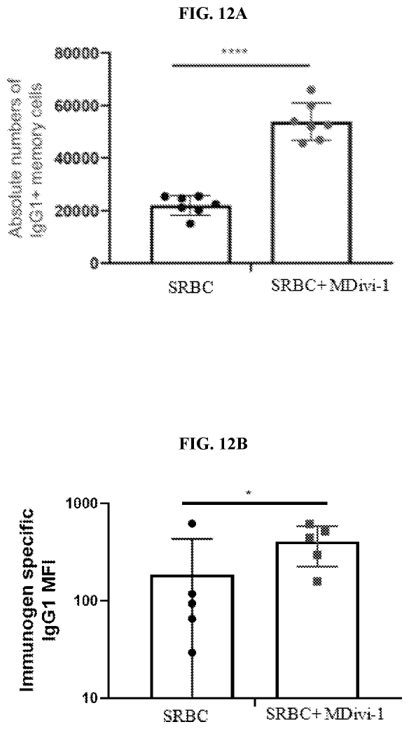

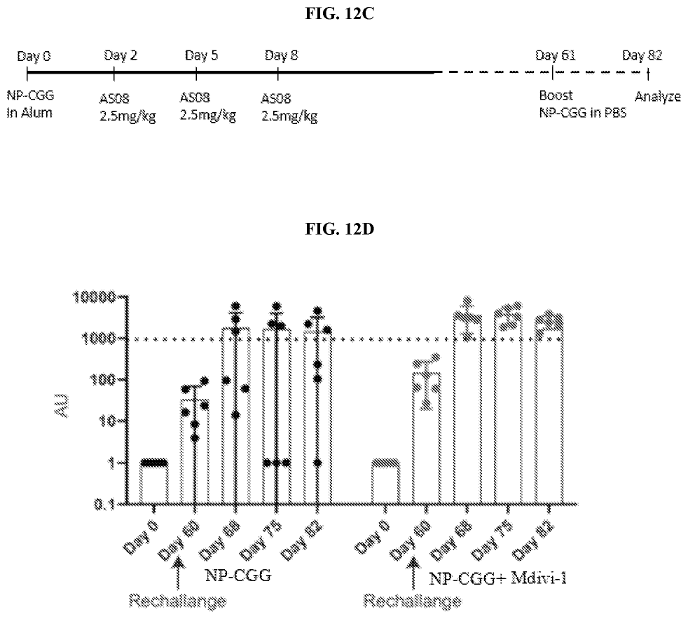

Seven mice were immunized with SRBC Day 0, and further injected with SBP-AS08 on day 2. Then, serum from immunized mice was incubated with SRBC and stained by anti-mouse IgG1 antibody to detect the SRBC specific IgG1 antibody. FIG. 12A is the graph showing absolute number of IgG1 memory cells in SRBC alone and SRBC with SBP-A508, indicating the increase of IgG1 memory cells in the mice injected with SBP-AS08. FIG. 12B shows a bar graph graph in log scale for SRBC specific IgG1 antibody in mice immunized with SRBC and SRBC with SP-AS08.

FIG. 12C is a schematic of an experimental design of multiple injections of Mdivi-1 (SBP-A508) for studying NP-CGG immunization along with or without SBP-A508 treatment. As shown, mice were injected with NP-CGG on day 0, and further injected with SBP-A508 on day 2, Day 5, and Day 8 at a dose of 2.5 mg/kg each. Then, a boost immunization (rechallenge) was given on day 61. Mice were bleed on day 82 for further analysis. FIG. 12D is a bar graph in log scale showing the NP-2 specific antibody production measured by ELISA in control and SBP-AS08 treated group upon rechallenge with NP-CGG in PBS on Day 61. Mice were bleed on Days 0, 60, 68, 75 and 82.

### Example 11

### Mdivi-1 Inhibitor Enhances Antigen Specific Response

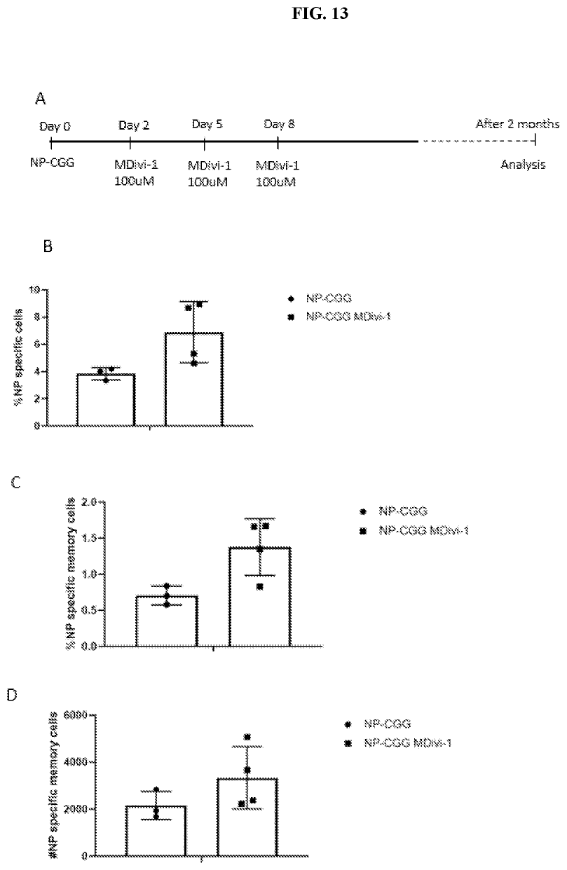

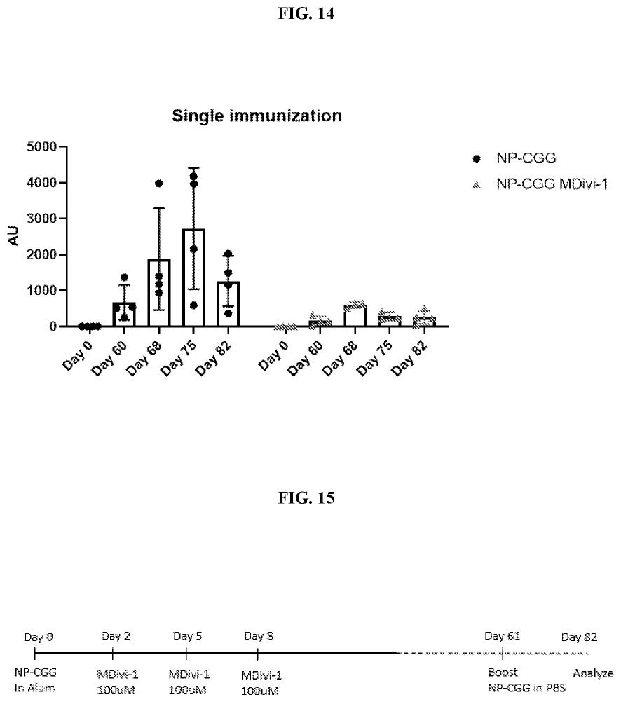

FIG. 13A depicts an experimental design for studying NP-CGG immunization along with or without Mdivi-1 treatment. Briefly, Mdivi-1 treatments were performed at least three times after immunization (day 2, day 5, and day 8), and the blood samples from the subject were analyzed at 2 months after the immunization. FIG. 13B shows a graph displaying the frequency of NP specific B cells in control and Mdivi-1 treated group, indicating that the substantial increase of NP specific B cell population with Mdivi-1 treatment. FIG. 13C shows a graph displaying frequencies NP specific memory B cells in control and Mdivi-1 treated group. FIG. 13D shows a graph displaying absolute numbers of NP specific memory B cells. Further, Mdivi-1 inhibitor treated mice exhibited increased NP specific antibody production. FIG. 14 shows a graph showing the NP specific antibody production measured by ELISA in control and Mdivi-1 treated group. Mice were bleed on days 0, 60, 68, 75 and 82. This example indicates that the effect of Mdivi-1 treatment in immune memory formation is antigen-specific.

### Example 12

### Mdivi-1 Inhibitor Generates True Memory Responses

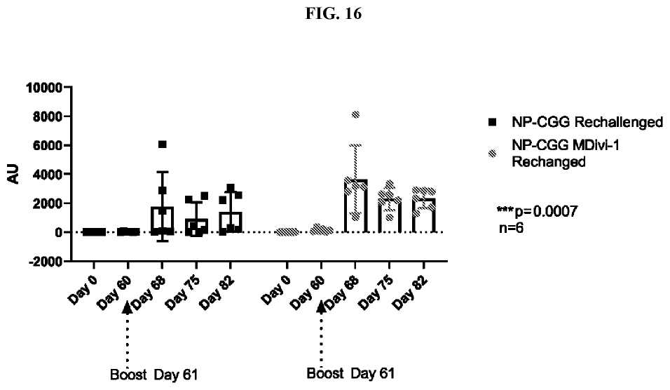

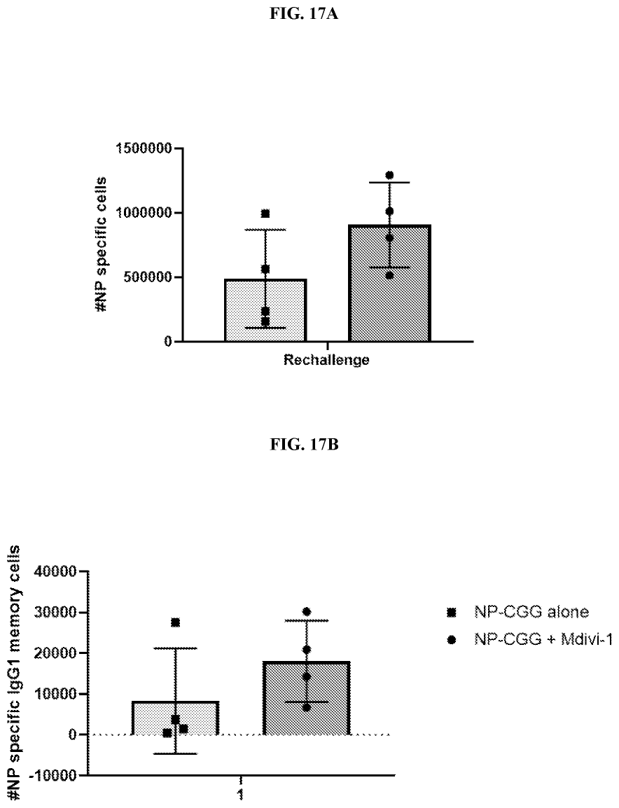

Mice were immunized with NP-CGG and treated with Mdivi-1 at least three times after immunization (day 2, day 5, and day 8). Then, a boost immunization (rechallenge) was given on day 61. Mice were bleed on day 82 for further analysis. FIG. 15 shows such immunization plan with NP-CGG to test if the memory generated is true. FIG. 16 shows a graph showing the NP specific antibody production measured by ELISA in control and Mdivi-1 treated group upon rechallenge with NP-CGG in PBS on Day 61. Mice were bleed on days 0, 60, 68, 75 and 82 after immunization with NP-CGG. It is noted that antigen specific antibody generation was substantially increased in Mdivi-1 inhibitor treated mice after antigen boost was provided. As shown in FIGS. 17A-B, Mdivi-1 treated mice replenished their memory B cell pool. The graph shown in FIG. 17A displays the absolute numbers of NP specific B cells in control and Mdivi-1 treated group. The graph shown in FIG. 17B shows absolute numbers of NP specific IgG1 memory B cells in control and Mdivi-1 treated group. In this experiment, the number of NP specific immune cells (left graph) as well as NP specific IgG1 memory cells were significantly increased or more in Mdivi-1 treated group.

### Example 13

### Mdivi-1 Treated Mice Have Enriched T Follicular Helper (TFh) Cells

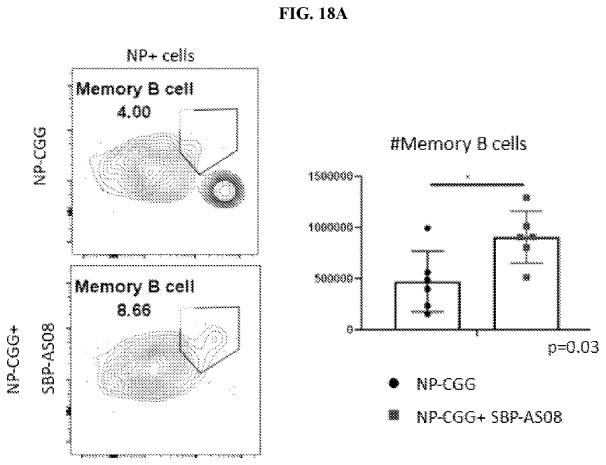

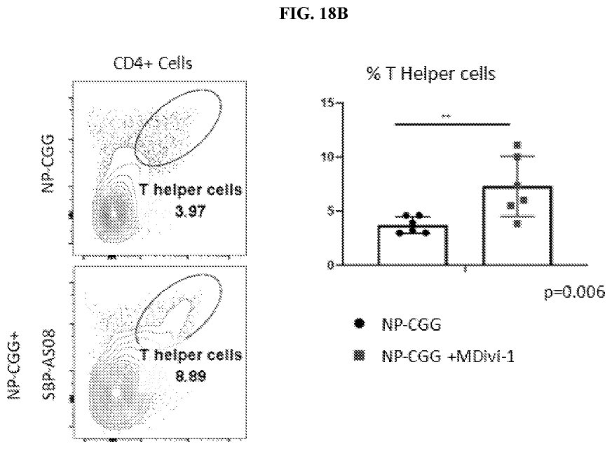

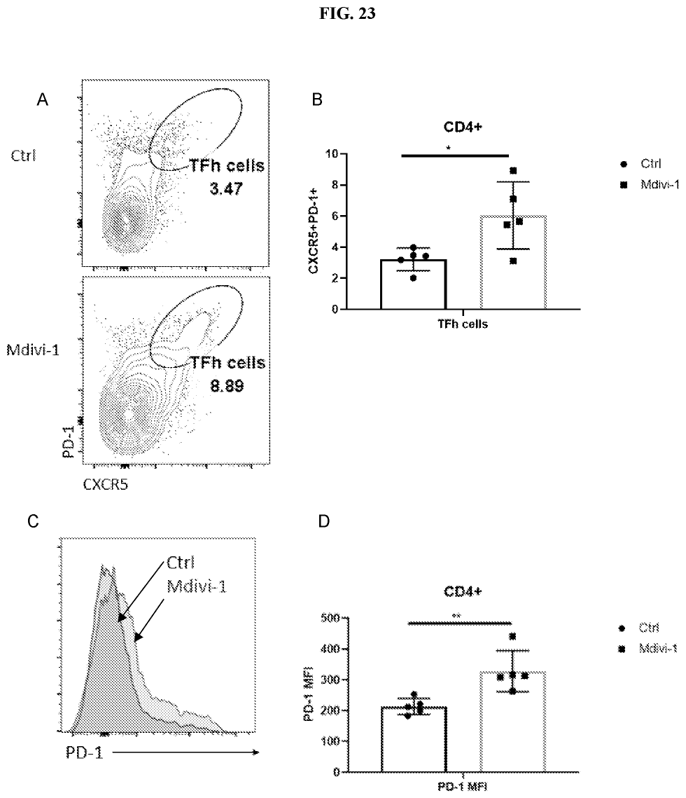

Germinal center B cells plays a major role in the proliferation of TFh cells. Dot plots shown in FIG. 18B show the frequencies of TFh cells. As shown in the graph in FIG. 18B, the frequency of TFh cells was substantially increased in Mdivi-1 treated group compared to control. Similarly, FIGS. 23A-B show flow plots (FIG. 23A) and graph (FIG. 23B) displaying enrichment of T follicular helper cells in mice treated with Mdivi-1. Further, as shown FIGS. 23C-D, PD-1 molecule expression on T follicular helper cells in Mdivi-1 treated group measured by median fluorescent intensity was higher than nontreated, control group, indicating that T follicular helper cells were activated upon Mdivi-1 treatment.

### Example 14

### Mdivi-1 Treated Mice Have Enriched IgG1 Memory Population

FIG. 20 shows an immunization plan to measure the memory precursor B cell population with or without Mdivi-1 treatment. Mice were immunized with Sheep red blood cells (SRBC) to mount an immune response. One group was treated with Mdivi-1 once at the dose of 2.5 mg/kg on Day 3. Blood samples of all groups were analyzed on day 11 after initial immunization.

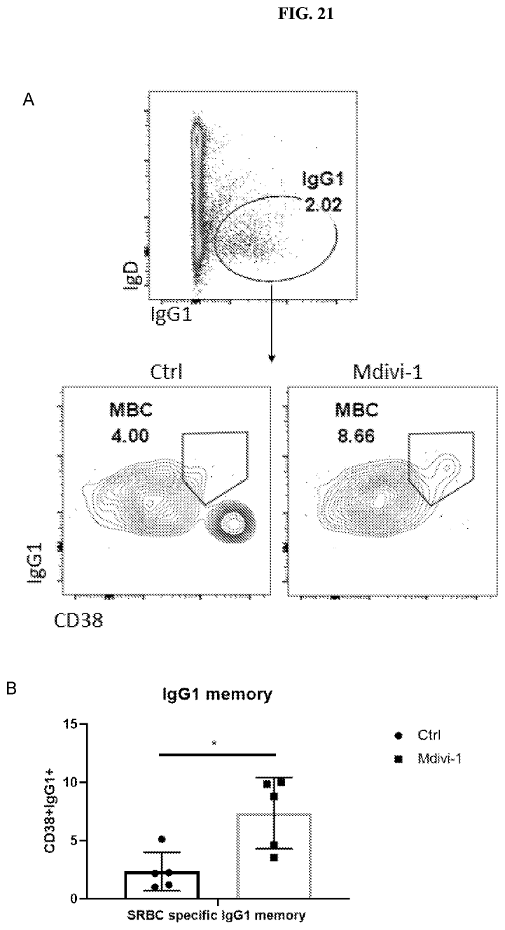

FIGS. 21A-B show increased IgG1 memory cell population upon Mdivi-1 treatment. FIG. 13A show flow plots showing IgG1 population and gating memory B cells in immunized control and Mdivi-1 treated mice. FIG. 21B shows a graph plot of frequencies of IgG1 memory B cells in control and Mdivi-1 treated immunized mice. It is noted that the IgG1+CD38+ cell population (IgG1 memory B cells: cells in the area marked with pentagons in the flow plots in FIG. 21A) were substantially increased with Mdivi-1 treatment.

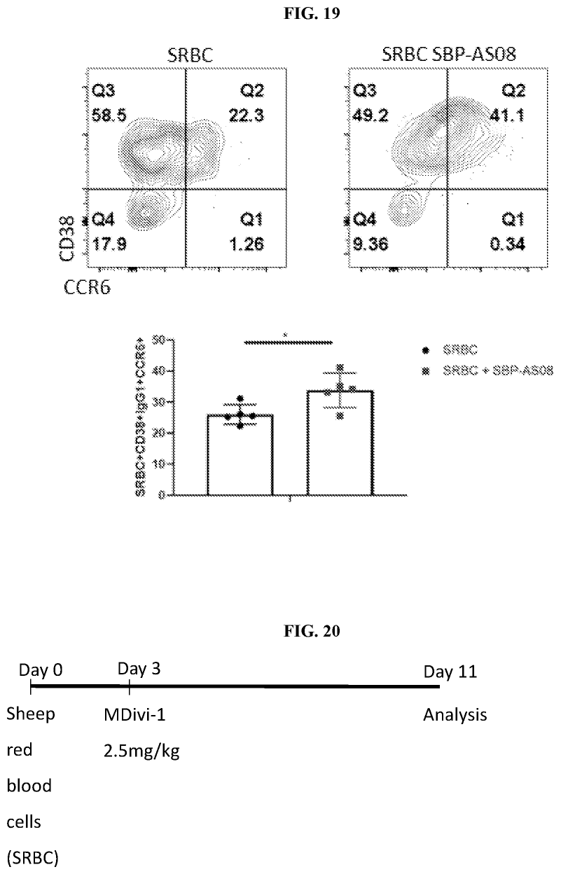

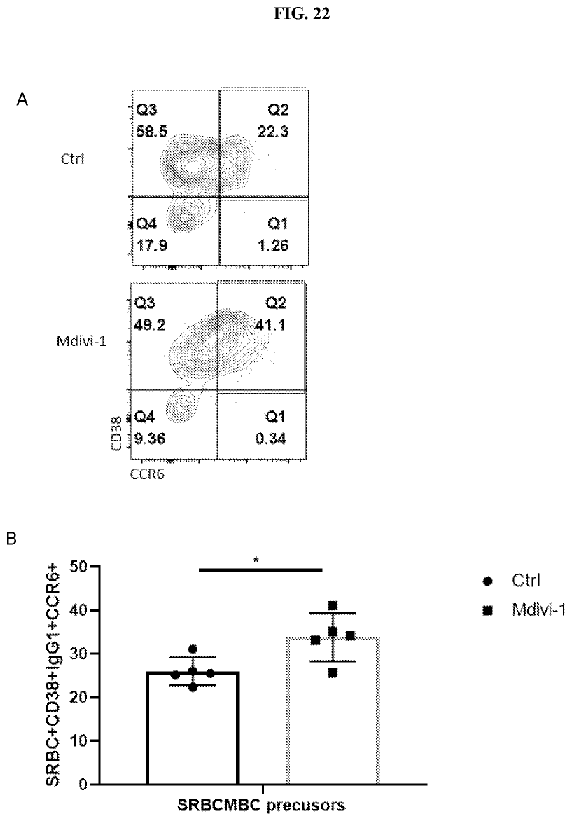

FIG. 19 shows flow plots showing population of SRBC-specific IgG1 memory precursors in ctrl and SBP-AS08 treated group. As shown in the bottom of the FIG. 19, frequencies of IgG1 memory precursors was significantly increased upon Mdivi-1 treatment. FIGS. 22A-B shows another data supporting increased memory precursors cells upon Mdivi-1 treatment. FIG. 22A show two flow plots showing population of SRBC-specific IgG1 memory precursors in control and Mdivi-1 treated group (right upper boxes). FIG. 22B is the graph showing frequencies of IgG1 memory precursors. It is noted that the IgG1+CD38+ cell population (memory precursors cells, cells in the right upper boxes of FIG. 22A)were substantially increased with Mdivi-1 treatment.

### Example 15

### Metabolic Alteration by Mdivi-1 Increases the Efficacy of Immunization

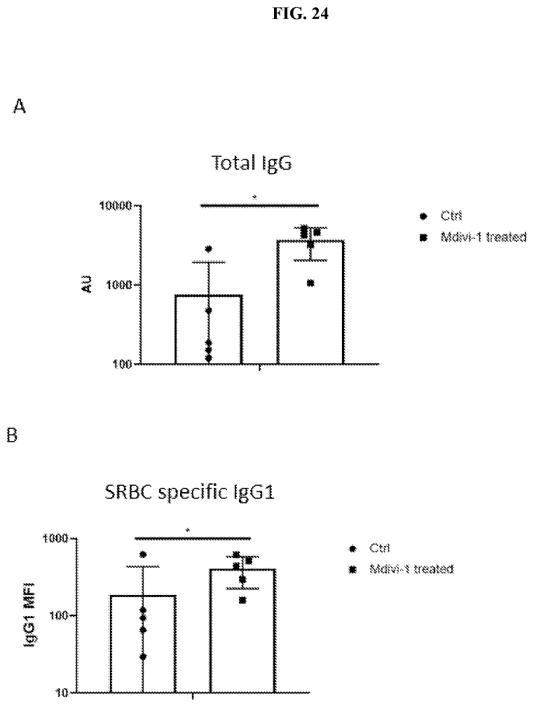

FIG. 24A shows a graph quantifying total IgG antibodies measured by ELISA with or without Mdivi-1 treatment. FIG. 24B shows a graph of SRBC specific IgG antibodies measured by ELISA. Sera was incubated with SRBC to bind all the SRBC specific antibody, followed by staining SRBCs with anti-IgG1 to determine the SRBC specific IgG1 in control and Mdivi-1 treated group of mice.

### Example 16

### Mdivi-1 Alters the Mitochondrial Organization Upon BCR Stimulation in B Cells

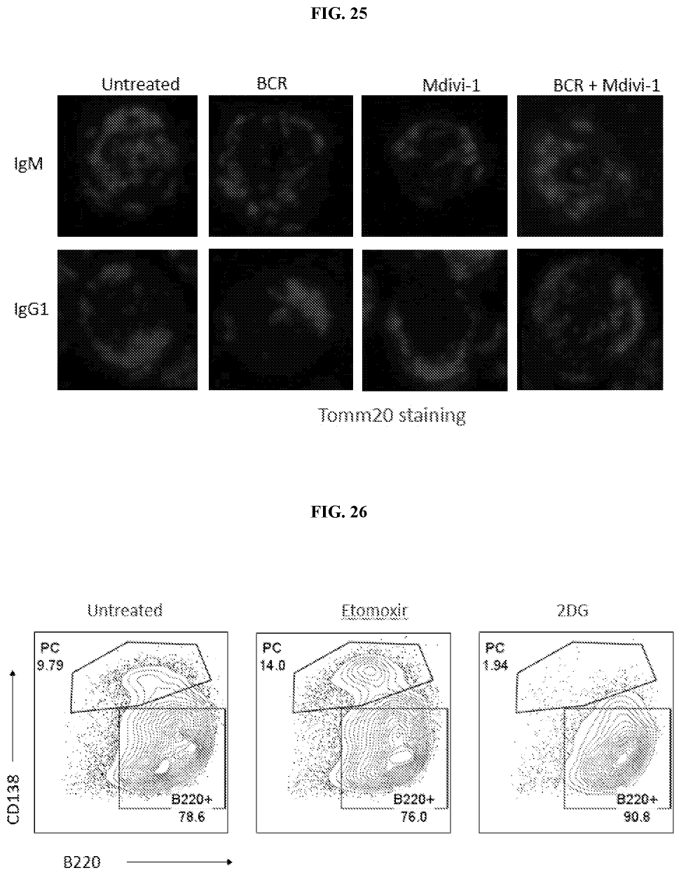

FIG. 25 shows confocal images of mitochondria organization in IgM and IgG1 cells at naïve stage (untreated), stimulated stage (BCR), Mdivi-1 treatment alone stage (Mdivi-1) and stimulation in presence of Mdivi-1 (BCR+Mdivi-1). The data indicates that Mdivi-1 treatment prevents the mitochondrial fission in BCR treated cells.

### Example 17

### Inhibition of Fatty Acid Oxidation Enhances the Plasma Cell Differentiation

FIG. 26 shows a data of the inhibition of fatty acid oxidation by Etomoxir enhances the plasma cells (PC) differentiation in vitro. Cells were treated either with Etomoxir to prevent fatty acid oxidation or with 2-deoxy-D-glucose (2DG) to facilitate the fatty acid oxidation to see the effect of fatty acid oxidation in plasma cell differentiation. Note that the number of CD138+ cells were substantially increased with Etomoxir treatment and the number of CD138+ cells were substantially decreased with 2DG treatment, indicating that inhibition of fatty acid oxidation enhances the plasma cell differentiation.

### Example 18

### Mdivi-1 Boosts the Protection Against Fatal Influenza Infection

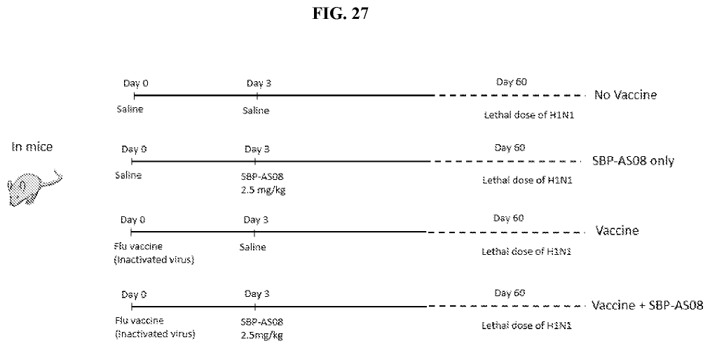

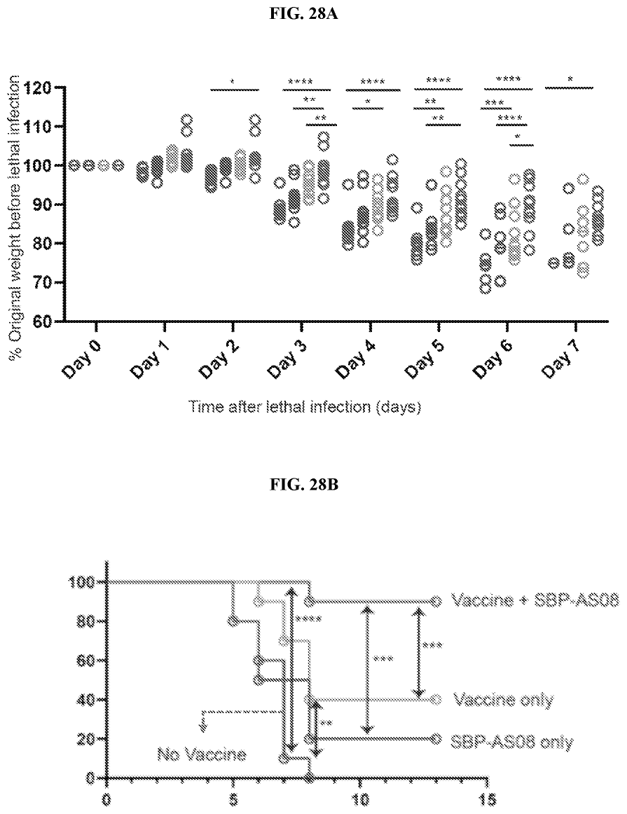

FIG. 27 shows a schematic of experimental design. 4 groups of ten mice each (6-8 weeks) were vaccinated intra-nasally with flu vaccine (vaccine group and vaccine+Mdivi-1 (SBP AS-08) group) or saline (No Vaccine group). In day 3, vaccine+Mdivi-1 (SBP AS-08) group and Mdivi-1 only group were administered with 2.5 mg/kg dose of Mdivi-1 (SBP AS-08), while all other groups were administered with saline. In day 60, all groups were administered with a lethal dose of H1N1. FIG. 28A is an individual dot graph showing the weight of mice in each group post lethal H1N1 viral infection. For each time point, dot or a group of dots from left to right represent the sample fo no vaccine, SBP-AS08 only, vaccine only, and vaccine and SBP-AS08, respectively. FIG. 28B is a Kaplan Meier event free survival curve, indicating that SBP-AS08 (MDIVI-1) boosts the protection against fatal influenza infection. Mice were sacrificed if the weight reached 70% of their original weight or appeared very sick. N=10 per group, *p=0.04, ** p=0.001, ***p=0.0001, ****p<0.0001.

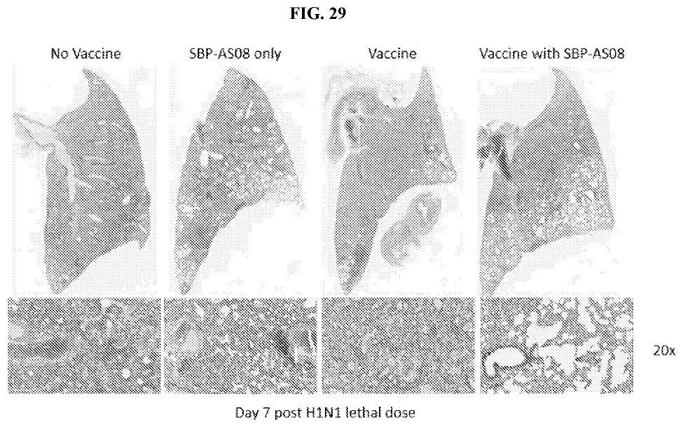

Mice from each group were also sacrificed on Day 7 post lethal influenza infection. FIG. 29 shows photographs of lung histology of sacrificed mice showing the degree infection, loss of lung morphology and alveolar structure using H&E stain, indicating that coadministration of SBP-AS08 reduces the disease progression post H1N1.

### Example 19

### Mdivi-1 Enhances the Immune Response in Older Age Mice

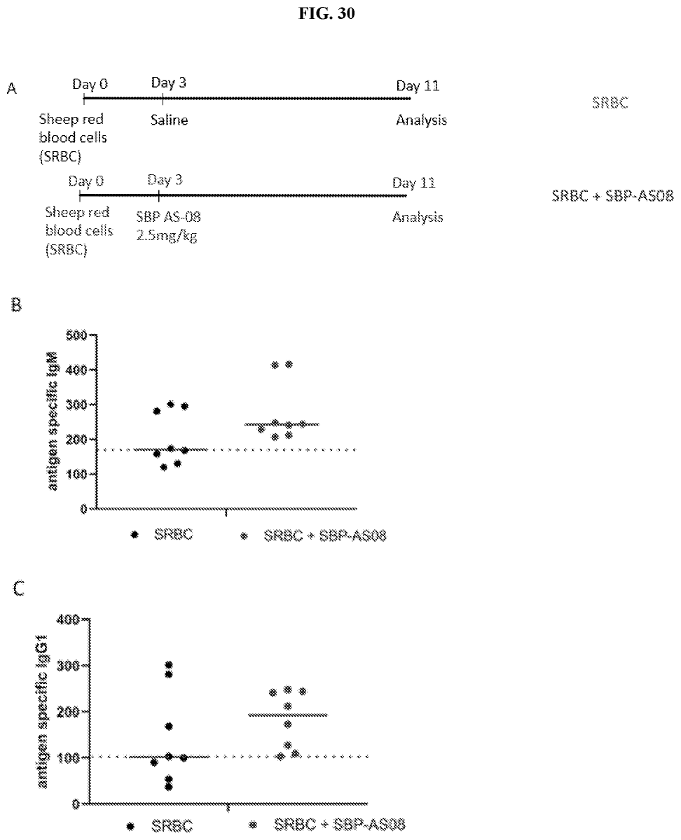

FIG. 30A shows a schematic of experimental design. 2 groups of eight mice each (14-24 months old age) were immunized intraperitoneally with sheep red blood cells (SRBC). In day 3, one group (SRBC group) was administered with saline, and another group (SRBC+Mdivi-1 (SBP-AS08)) was administered with 2.5 mg/kg dose of Mdivi-1 (SBP AS-08). FIG. 30B and FIG. 30C show scatter plots of SRBC specific anti-IgM antibody or SRBC specific anti-IgG1 antibody of two groups in day 11 after administration of SRBC, respectively. Each dot represents each individual mice in each group. As shown, SRBC specific anti-IgG1 antibody is substantially increased in SRBC+Mdivi-1 (SBP-AS08) group compared to the SRBC group.

### Example 20

### Mdivi-1 Enhances Covid-19 Vaccine Efficacy

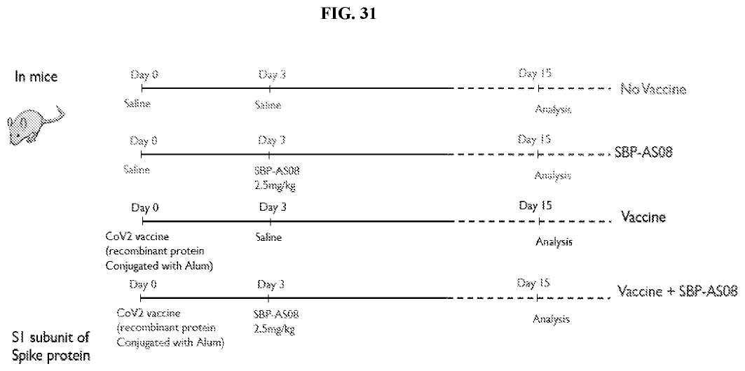

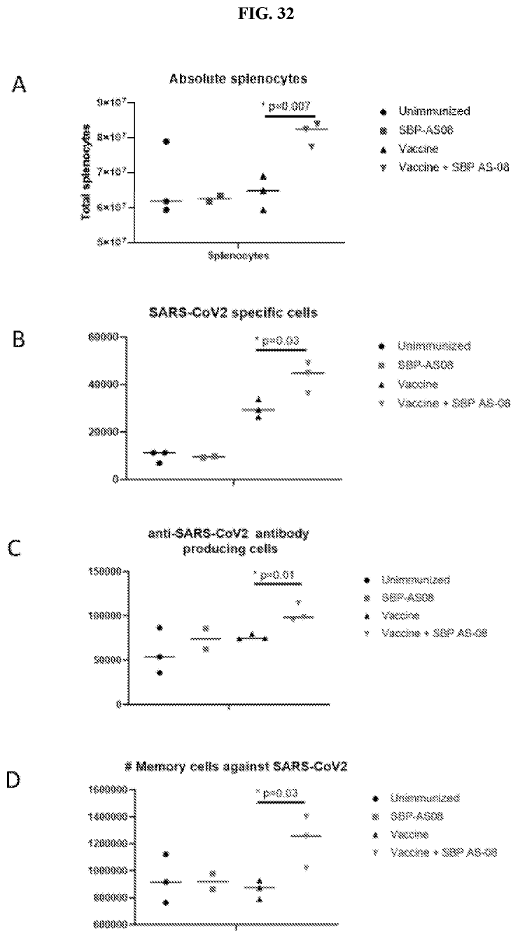

Effect of single injection of Mdivi-1 (SBP-AS08): The efficacy of Mdivi-1 in SARS-Covid19 vaccines was tested. FIG. 31 illustrate a schematic of SARS-CoV2 experiment. In this experiment, a recombinant protein of Si subunit of corona virus spike protein conjugated with an adjuvant Alum was used as a CoV2 vaccine. Mice were immunized with S1 subunit of corona virus spike protein conjugated with alum. On Day 3 post immunization, half of mice were administered with Mdivi-1 (SBP-A508) at a dose of 2.5mg/kg. On Day 15, mice were sacrificed and were analyzed. FIGS. 32A-D show plots of cell number counts of various cell types in each control and experimental groups. FIG. 32A shows the absolute numbers of splenocytes from each group of mice counted by automated cell counter. Cells were incubated with spike protein and stained with anti-His tag to detect SARS-CoV2 specific cells. Cells were further stained with markers specific for B cells memory B cells and antibody producing plasma cells. Cellular population was analyzed by flow cytometry, and the numbers of SARS-CoV2-specific cells, anti-SARS-CoV2 antibody producing cells, and SARS-CoV2-specific memory B cells are plotted in FIG. 32B, FIG. 32C, and FIG. 32D, respectively. Collectively, the number of splenocytes, SARS-CoV2-specific cells, anti-SARS-CoV2 antibody producing cells, and SARS-CoV2-specific memory B cells are increased in mice administered with Mdivi-1 after SARS-CoV2 vaccination, indicating that Mdivi-1 enhances the efficacy of the SARS-Covid19 vaccines.

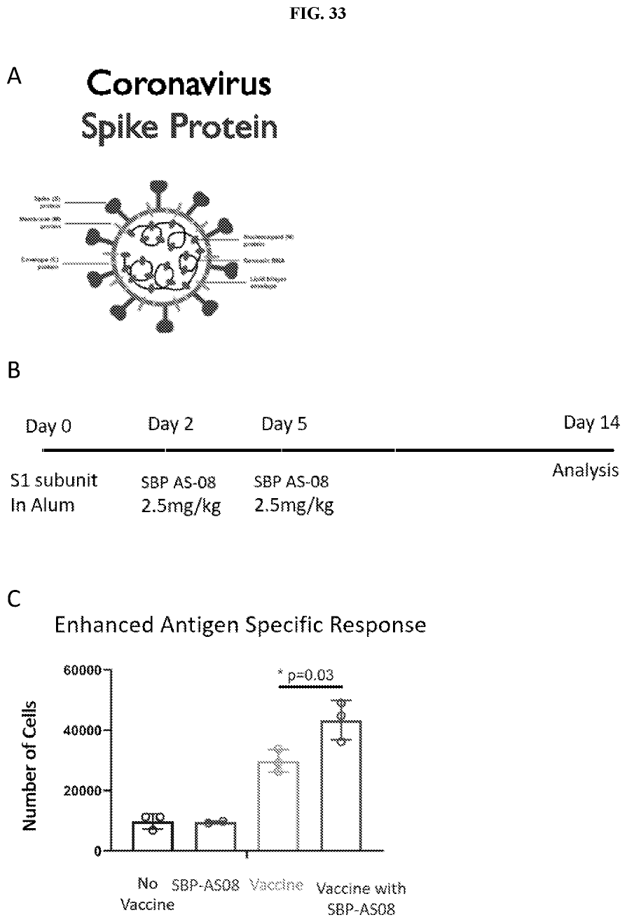

Effect of multiple injections of Mdivi-1 (SBP-A508): In this experiment, a recombinant protein of S1 subunit of spike protein conjugated with an adjuvant alum was used as a vaccine. A schematic illustration of corona virus and its spike proteins is shown in FIG. 33A. FIG. 33B depicts a schematic of experimental design. Mice were immunized with S1 subunit of coronavirus spike protein conjugated with alum. Day 2 and Day 5 post immunization half of mice were given SBP-AS08. On Day 15 mice were sacrificed and were analyzed. Cells were incubated with spike protein and stained with anti-His tag to detect SARS-CoV2 specific cells. Cells were further stained with markers specific for B cells memory B cells and antibody producing plasma cells. Cellular population was analyzed by flow cytometry. As shown in FIG. 33C, the number of marker-specific B cells is significantly increased in a group treated with vaccine, and further increased in a group treated with vaccine with Mdivi-1 (SBP-AS08), indicating that Mdivi-1 enhances the efficacy of the SARS-Covid19 vaccines.

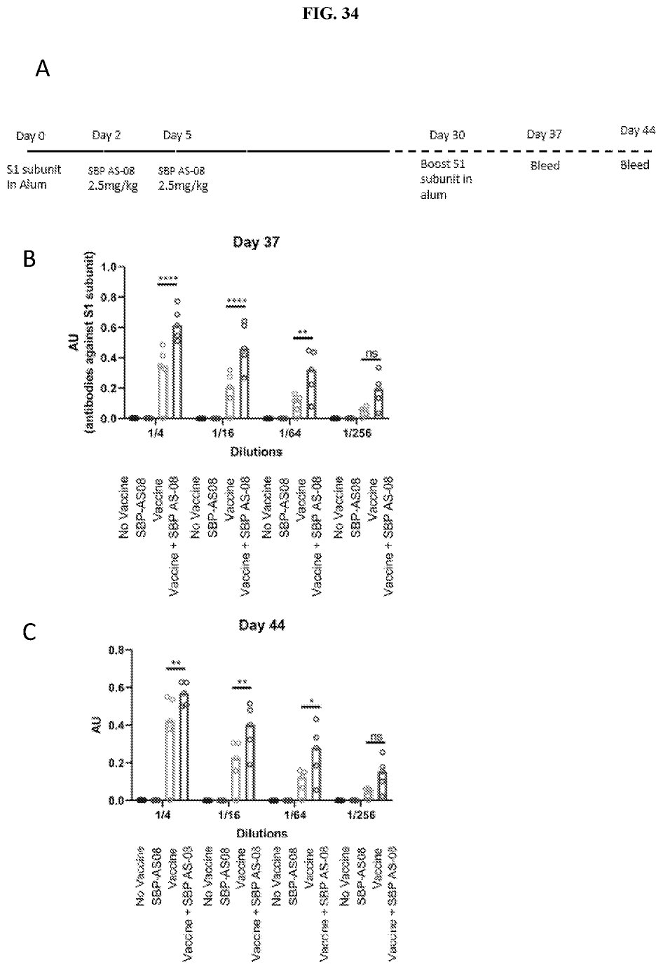

Effect of multiple injections of Mdivi-1 (SBP-AS08) after rechallenge: In this experiment, a recombinant protein of S1 subunit of spike protein conjugated with an adjuvant alum was used as a vaccine. A schematic illustration of corona virus and its spike proteins is shown in FIG. 34A. Mice were immunized with S1 subunit of coronavirus spike protein conjugated with alum. Day 2 and Day 5 post immunization half of mice were given SBP-AS08 at a dose of 2.5 mg/kg. A boost immunization (rechallenge) was given on day 30, and mice were bled after on day 37 and Day 44. FIG. 34B and FIG. 34C show bar graphs of the S1 specific antibody titers in serum from 4 groups of mice on Day 37 and Day 44, indicating that Mdivi-1 enhances the efficacy of the SARS-Covid19 vaccines after rechallenge.

### Example 21

### Surrogate COVID-19 Vaccine with SBP-AS08

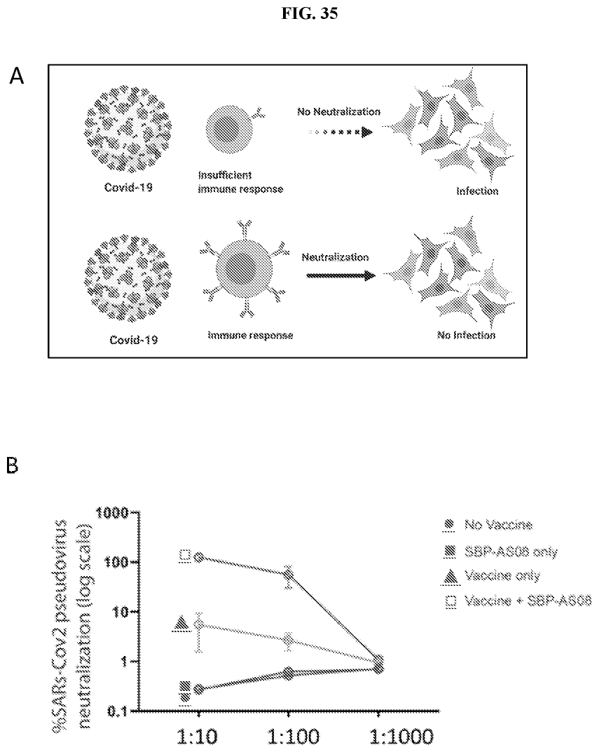

The strength of vaccine can be determined by the percentage or number of neutralizing antibodies that act against the antigen-containing organism after the vaccine administration. For example, where the immune response is insufficient, no neutralization effect would occur upon the infection, while where the immune response is sufficient or strong enough by the vaccine, the immune system effectively neutralize the infection. (FIG. 35A) In this experiment, serum collected from mice on Day 37 (in the experiment of Example 19) was incubated with pseudo SARS-CoV2 virus for one hour at 37 C and then human Vero cells expressing ace-2 receptor were infected the mixture. Luciferase assay was performed as a neutralization assay on 48 hours post infection. FIG. 35B shows a line graph of % of SARs-Cov2 pseudovirus neutralization in log scale, indicating higher % of neutralizing antibody titers in Mdivi-1 treated mice serum.

### Example 22

### Developing Novel Neo-Antigen Vaccine Based Therapy for Pancreatic Cancer Using SBP-AS08

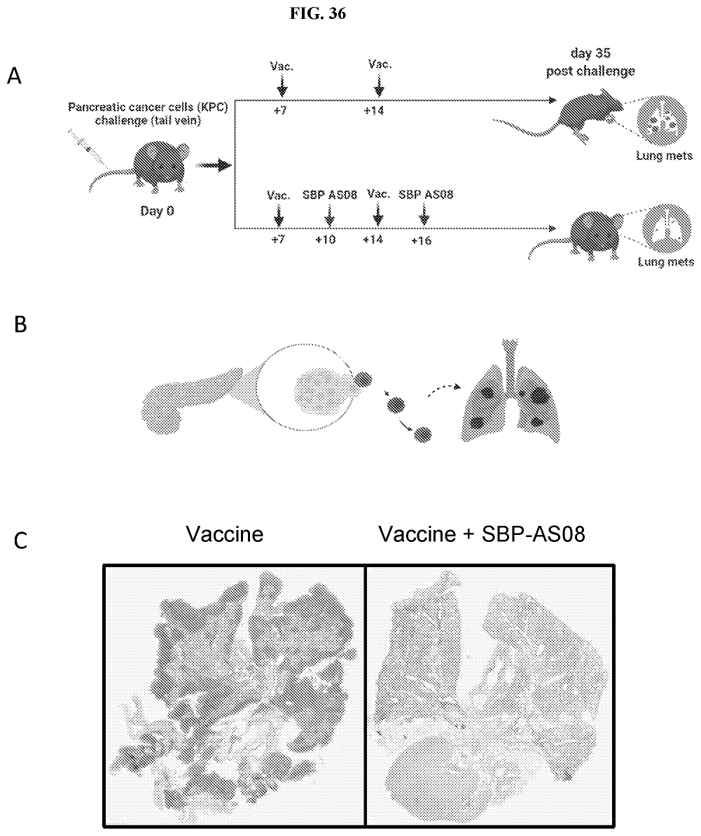

The effect of Mdivi-1 (SBP-AS08) in boosting immunity can be extrapolated to other diseases related to specific immune response (e.g., cancer, etc.). FIG. 36A is a schematic of an experimental layout to test the effect of Mdivi-1 (SBP-AS08) in mouse cancer model. In this experiment, mouse pancreatic cancer cell line lysate was used as the vaccine. 200,000 mouse pancreatic cancer cells were injected in 10 (C57/B6) mice per group via tail vein. Effectiveness of the vaccine was tested by analyzing the metastasis of the cancer cells from the pancreas to lungs (FIG. 36B). Lungs tissues were collected from group of vaccine and vaccine with SBP-AS08 on Day 22 post injections. Two more groups with no treatment and only SBP-AS08 treatment were also included in the analysis. FIG. 36C shows H&E stained section of whole lungs showing metastasis stained by dark purple color in mice from vaccine and vaccine with SBP-AS08.

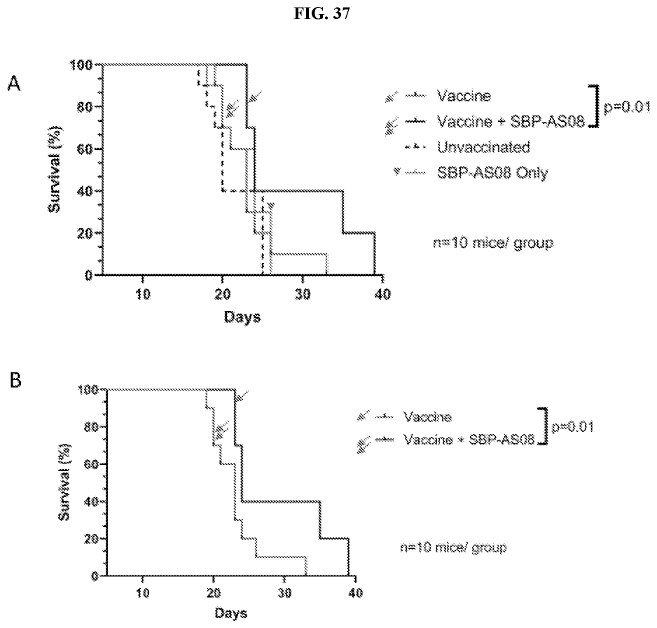

FIG. 37A and FIG. 37B show Kaplan Meier survival curve of mice that are unvaccinated, treated with SBP-AS08 only, treated with vaccine, or treated with vaccine and SBP-AS08. As shown, neo-antigen vaccine with SBP-AS08 increases the survival of tumor bearing mice.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of increasing the effectiveness of a vaccine in a subject, comprising:
administering a B cell metabolic reprogramming agent to the subject in a dose and schedule configured to increase the effectiveness of the vaccine, wherein the subject is administered with the vaccine.

2. The method of claim 1, wherein the B cell metabolic reprogramming agent is an agent increasing mitochondrial mass or enhancing mitochondrial function.

3. The method of claim 2, wherein the agent increasing mitochondrial mass or enhancing mitochondrial function is a Drp1 inhibitor.

4. The method of claim 3, wherein the Drp1 inhibitor is Mdivi-1, dynasore, or dyngo 4a.

5. The method of claim 4, wherein the dose is between 1.0-50.0 mg/kg.

6. The method of any one of claims 4-5, wherein the dose is about 2.5 mg/kg.

7. The method of any one of claims 1-6, wherein the B cell metabolic reprogramming agent is administered concurrently with the vaccine.

8. The method of claim 7, wherein the B cell metabolic reprogramming agent is an immune enhancer for the vaccine.

9. The method of any one of claims 1-8, wherein the B cell metabolic reprogramming agent is administered at least a day after administering the vaccine.

10. The method of any one of claims 1-9, wherein the B cell metabolic reprogramming agent is administered at least 2 days after administering the vaccine.

11. The method of any one of claims 1-10, wherein the B cell metabolic reprogramming agent is administered a plurality of times in a regular interval after administering the vaccine.

12. The method of any one of claims 1-11, wherein the effectiveness of the vaccine is increased by inhibiting mitochondrial mass decrease in B cells or other immune cells.

13. The method of any one of claims 1-12, wherein the effectiveness of the vaccine is increased by increasing memory B cell population in the subject.

14. The method of any one of claims 1-13, wherein the effectiveness of the vaccine is increased by increasing memory B cell precursor population in the subject.

15. The method of any one of claims 1-14, wherein the effectiveness of the vaccine is increased by increasing replenishment of memory B cell population in the subject after rechallenge.

16. The method of claim 15, wherein the memory B cell population comprises IgG cells.

17. The method of any one of claims 1-16, wherein the effectiveness of the vaccine is increased by increasing TFh cell population in the subject after rechallenge.

18. The method of any one of claims 1-17, wherein the dose and schedule is sufficient to increase antigen-specific antibody titers at least 50% in the subject compared to a subject not receiving the B cell metabolic reprogramming agent after administering the vaccine.

19. The method of any one of claims 1-18, wherein the dose and schedule is sufficient to increase antigen-specific antibody titers at least 50% in the subject compared to a subject not receiving the B cell metabolic reprogramming agent after rechallenge.

20. The method of any one of claims 1-19, wherein the dose and schedule is sufficient to prevent decreased oxygen consumption of mitochondria in IgG cells in the subject.

21. The method of any one of claims 1-20, wherein the vaccine comprises a live-attenuated vaccine, an inactivated vaccine, a recombinant vaccine, a conjugate vaccine, a polysaccharide, a DNA-based vaccines, an RNA-based vaccines, or a toxoid vaccine.

22. The method of any one of claims 1-21, wherein the vaccine comprises an influenza vaccine or a SARS-CoV2 vaccine.

23. A method of increasing immunity against an antigen in a subject having an immune response against the antigen, comprising:
administering a B cell metabolic reprogramming agent to the subject in a dose and schedule configured to increase a secondary immune response upon re-exposure to the antigen compared to a subject not being administered with the B cell metabolic reprogramming agent.

24. The method of claim 23, wherein B cell metabolic reprogramming agent is an agent increasing mitochondrial mass or enhancing mitochondrial function.

25. The method of claim 24, wherein the agent increasing mitochondrial mass or enhancing mitochondrial function is a Drp1 inhibitor.

26. The method of claim 25, wherein the Drp1 inhibitor is Mdivi-1, dynasore, or dyngo 4a.

27. The method of claim 26, wherein the dose is between 1.0-50.0 mg/kg.

28. The method of any one of claims 26-27, wherein the dose is about 2.5 mg/kg.

29. The method of any one of claims 23-28, wherein the B cell metabolic reprogramming agent is administered during the immune response.

30. The method of any one of claims 23-29, wherein the schedule comprises administration at least a day after the immune response.

31. The method of any one of claims 23-30, wherein the schedule comprises administration at least 2 days after the immune response.

32. The method of any one of claims 23-31, wherein the schedule comprises administration a plurality of times in a regular interval after the immune response.

33. The method of any one of claims 23-32, wherein the dose and schedule is sufficient to inhibit mitochondrial mass decrease in B cells or other immune cells of the subject.

34. The method of any one of claims 23-33, wherein the dose and schedule is sufficient to increase memory B cell population in the subject.

35. The method of any one of claims 23-34, wherein the dose and schedule is sufficient to increase memory B cell precursor population in the subject.

36. The method of any one of claims 23-35, wherein the memory B cell population is increased by facilitating replenishment of memory B cells in the subject after the re-exposure to the antigen.

37. The method of claim 36, wherein the memory B cell population comprises IgG cells.

38. The method of any one of claims 23-37, wherein the memory B cell population is increased by increasing TFh cell population in the subject after re-exposure to the antigen.

39. The method of any one of claims 23-38, wherein the dose and schedule is sufficient to increase antigen-specific antibody titers at least 50% in the subject compared to a subject not receiving the mitochondria fission inhibitor after re-exposure to the antigen.

40. The method of any one of claims 23-39, wherein the dose and schedule is sufficient to prevent decreased oxygen consumption of mitochondria in IgG1 positive cells in the subject.

41. A method of increasing a memory B cell population in a subject having an immune response against the antigen, comprising:
administering a B cell metabolic reprogramming agent to the subject in a dose and schedule configured to increase the memory B cell population after exposure to the antigen compared to a subject not being administered with the agent.

42. The method of claim 41, wherein B cell metabolic reprogramming agent is an agent increasing mitochondrial mass or enhancing mitochondrial function.

43. The method of claim 42, wherein the agent increasing mitochondrial mass or enhancing mitochondrial function is a Drp1 inhibitor.

44. The method of claim 43, wherein the Drp1 inhibitor is Mdivi-1, dynasore, or dyngo 4a.

45. The method of claim 44, wherein the dose is between 1.0-50.0 mg/kg.

46. The method of any one of claims 43-44, wherein the dose is about 2.5 mg/kg.

47. The method of any one of claims 41-46, wherein the B cell metabolic reprogramming agent is administered during the immune response.

48. The method of any one of claims 41-47, wherein the schedule comprises administration at least a day after the immune response.

49. The method of any one of claims 41-48, wherein the schedule comprises administration at least 2 days after the immune response.

50. The method of any one of claims 41-49, wherein the schedule comprises administration a plurality of times in a regular interval after the immune response.

51. The method of any one of claims 41-50, wherein the dose and schedule is sufficient to inhibit mitochondrial mass decrease in B cells or other immune cells of the subject.

52. The method of any one of claims 41-51, wherein the dose and schedule is sufficient to increase memory B cell precursor population in the subject.

53. The method of any one of claims 41-52, wherein the memory B cell population is increased by facilitating replenishment of memory B cells in the subject after the re-exposure to the antigen.

54. The method of any one of claims 41-53, wherein the memory B cell population comprises IgG cells.

55. The method of any one of claims 41-54, wherein the memory B cell population is increased by increasing TFh cell population in the subject after re-exposure to the antigen.

56. The method of any one of claims 41-55, wherein the dose and schedule is sufficient to increase antigen-specific antibody titers at least 50% in the subject compared to a subject not receiving the mitochondria fission inhibitor after re-exposure to the antigen.

57. The method of any one of claims 41-56, wherein the dose and schedule is sufficient to prevent decreased oxygen consumption of mitochondria in IgG1 positive cells in the subject.

58. A pharmaceutical composition, comprising: a vaccine composition and an agent that triggers metabolic reprogramming of B cells, wherein the agent is present in the composition in a dose effective to increase effectiveness of the vaccine.

59. The pharmaceutical composition of claim 58, wherein the agent a mitochondria fission inhibitor.

60. The pharmaceutical composition of claim 59, wherein the mitochondria fission inhibitor a Drp1 inhibitor.

61. The pharmaceutical composition of claim 60, wherein the Drp1 inhibitor is Mdivi-1, dynasore, or dyngo 4a.

62. The pharmaceutical composition of claim 61, wherein the dose is between 1.0-50.0 mg/kg.

63. The pharmaceutical composition of any one of claims 60-62, wherein the dose is about 2.5 mg/kg.

64. The pharmaceutical composition of any one of claims 58-63, wherein the agent is an immune enhancer for the vaccine.

65. The pharmaceutical composition of any one of claims 58-64, wherein the dose is effective to inhibit mitochondrial mass decrease in B cells or other immune cells in a subject when administered.

66. The pharmaceutical composition of any one of claims 58-65, wherein the dose is effective to increase memory B cell population in a subject when administered.

67. The pharmaceutical composition of any one of claims 58-66, wherein the dose is effective to increase memory B cell precursor population in a subject in a subject when administered.

68. The pharmaceutical composition of any one of claims 58-67, wherein the dose of is effective to increase replenishment of memory B cell population in the subject after rechallenge.

69. The pharmaceutical composition of claim 68, wherein the memory B cell population comprises IgG cells.

70. The pharmaceutical composition of any one of claims 58-69, wherein the dose is effective to increase TFh cell population in the subject after rechallenge.

71. The pharmaceutical composition of any one of claims 58-70, wherein the dose is effective to increase antigen-specific antibody titers at least 50% in a subject when administered, compared to a subject not receiving the composition.

72. The pharmaceutical composition of any one of claims 58-71, wherein the dose is effective to prevent decreased oxygen consumption of mitochondria in IgG cells in a subject when administered.

73. The pharmaceutical composition of any one of claims 58-72, wherein the vaccine composition comprises is a live-attenuated vaccine, an inactivated vaccine, a recombinant vaccine, a conjugate vaccine, a polysaccharide, a DNA-based vaccines, an RNA-based vaccines, or a toxoid vaccine.

74. The pharmaceutical composition of any one of claims 58-73, wherein the vaccine composition comprises an influenza vaccine or a SARS-CoV2 vaccine.

75. A pharmaceutical composition comprising i) a substance stimulating antibody production in a subject to provide immunity associated with a disease and ii) an agent increasing mitochondrial mass or enhancing mitochondrial function.
